(19) United States
(12) Patent Application Publication
(10) Pub. No.: US 2024/0352514 A1
(43) Pub. Date: Oct. 24, 2024

(54) METHOD AND SYSTEM FOR IDENTIFYING GENOMIC REGIONS WITH CONDITION SENSITIVE OCCUPANCY/POSITIONING OF NUCLEOSOMES AND/OR CHROMATIN

(71) Applicant: University of Essex Enterprises Limited, Colchester, Essex (GB)

(72) Inventor: Vladimir TEIF, Colchester, Essex (GB)

(21) Appl. No.: 18/563,324

(22) PCT Filed: May 23, 2022
(86) PCT No.: PCT/GB2022/051298

§ 371 Date: Nov. 21, 2023

(30) Foreign Application Priority Data
May 24, 2021 (GB) 2107400.0
May 25, 2021 (GB) 2107430.7

(51) Int. Cl. C12Q 1/6869

(52) U.S. Cl. CPC C12Q 1/6869

(57) ABSTRACT

Aspects of the present invention relate at least in part to identification of regions within the genome that are sensitive to a condition. Particularly, although not exclusively, embodiments of the present invention relate to a method and system for identifying regions of the genome where DNA protection from digestion changes in response to a condition, e.g. the nucleosomal organisation is different as compared to a genomic region in a subject without the condition. The condition may be a pathological disorder e.g. cancer, or a variation of a healthy state e.g. depending on person's lifestyle or age. In certain embodiments, the method and systems may identify regions within the genome which differ in patients with sub-sets of the same condition. Aspects of the present invention comprise identification, stratification and monitoring of subjects suffering from a condition by sequencing predetermined regions of a genome.

### FIELD OF THE INVENTION

Aspects of the present invention relate at least in part to identification of regions within the genome that are sensitive to a condition. Particularly, although not exclusively, embodiments of the present invention relate to a method and system for identifying regions of the genome where DNA protection from digestion changes in response to a condition, e.g. the nucleosomal organisation is different as compared to a genomic region in a subject without the condition. The condition may be a pathological disorder e.g. cancer, or a variation of a healthy state e.g. depending on person's lifestyle or age. In certain embodiments, the method and systems may identify regions within the genome which differ in patients with sub-sets of the same condition. Aspects of the present invention comprise identification, stratification and monitoring of subjects suffering from a condition by sequencing predetermined regions of a genome.

### BACKGROUND TO THE INVENTION

The “liquid biopsy” is one of the most promising methods of sampling for the early diagnostics of tumours and many other medical conditions, because it avoids invasive procedures such as tissue biopsies. This diagnostic approach is based on the analysis of disease-associated biomarkers in the blood plasma, urine or other body fluids. For example, circulating cell-free DNA (cfDNA) is present in liquid biopsies and consequently the experimental procedure of cfDNA extraction is relatively simple, especially compared to the procedures of more traditional DNA extracting biopsies.

At present, liquid biopsy assays based on next generation sequencing of cell-free DNA (cfDNA) are a promising strategy for screening, diagnostics, as well as patient monitoring and stratification. Such assays have diverse applications including prenatal testing, cancer and ageing. Several liquid biopsy assays have already been approved for clinical use, and more assays are expected to enter this rapidly growing market. Unfortunately, while there are many ongoing efforts to utilise cfDNA more routinely in clinical applications, there are a number of bottlenecks in respect of the computational analysis as well as cfDNA assay types, with current assays being predominantly based on DNA mutation or DNA methylation analysis. Such analysis methodologies are less suitable for early disease detection and may be limited to detecting established disease-specific changes. Many existing methods also require deep-sequencing of cfDNA, e.g. via whole-genome sequencing (WGS), whole-exome sequencing (WES) or bisulfite sequencing, which while very informative is costly.

Conversely, the use of more cost-effective shallow or moderate whole-genome sequencing may not provide enough sequencing coverage to robustly detect mutations or DNA methylation changes. This is especially challenging in early disease stages where the amount of disease-specific cfDNA is inevitably low, with this early stage also being the opportune time for diagnosis with regards to a patient's chance for curative treatment. It has been reported that elevated cfDNA levels correlate with all-causes mortality and so many assays use cfDNA concentration as a marker of disease severity without sequencing, yet this has clear limitations in respect of the specificity required for some uses (e.g., diagnostics). As a result, assay sensitivity critically depends on the sequencing depth as well as on the abundance of cfDNA derived from diseased cells (e.g. circulating tumour DNA (ctDNA)), with early diagnosis requiring deep-sequencing to offset the reduced abundance of cfDNA. Consequently, the mass-use of liquid biopsy assays as a standard clinical tool is dependent on finding novel methods that balance sensitivity and cost.

Thus, there is a clear need to develop cfDNA analysis methods that are not limited to the analysis of DNA mutations and epi-mutations, but focus on the analysis of cfDNA fragments per se (their properties and genomic locations of origin). Several methods of such computational analysis have been suggested previously, collectively termed “fragmentomics” or “nucleosomics”, including analyses such as the distribution of cfDNA fragment sizes; the density of cfDNA fragments in gene promoters; the 10-bp periodicity in cfDNA digestion sites arising from the periodicity in nucleosome organisation; and related methods. However, so far none of these methods have provided the sensitivity and/or specificity required for widespread clinical use. One of the reasons for the latter is that genome-wide (or exome-wide) analysis at a moderate sequencing depth contains a lot of non-specific noise and it is challenging to decipher condition-specific signal.

It is an aim of certain embodiments of the present invention to at least partially mitigate the problems associated with the prior art.

It is an aim of certain embodiments of the present invention to provide a method to define condition-sensitive genomic regions for the use in a liquid biopsy.

It is an aim of certain embodiments of the present invention to provide a liquid biopsy assay based on targeted genomic sequencing of condition-sensitive regions.

### SUMMARY OF CERTAIN EMBODIMENTS OF THE INVENTION

There remains a clear need to develop a method to define condition-sensitive genomic regions present in cell-free nucleic acids that are assessed in liquid biopsies. The development of such a method would be of great value in expanding the use of liquid biopsy assays into a standard clinical tool for a wide range of medical conditions, and so would be beneficial in early diagnostics, patient monitoring and stratification. In fact, assessment of the identified condition-sensitive genomic regions may form part of a liquid biopsy assay, which may then be used to diagnose, monitor a treatment response, or stratify a patient or a healthy person.

Certain embodiments of the present invention may provide assays based on the detection of small but statistically significant changes at predefined genomic loci, thereby solving the noise problem of genome-wide assays, and also the problem of developing more affordable assays based on targeted genomic sequencing of sensitive regions. Such a method may have value in developing liquid biopsy assays that are both cost-effective and sensitive, and so can be used as an effective clinical tool across a wide range of medical conditions.

In a first aspect of the present invention there is provided a method for identifying genomic regions with condition-specific protection, which have condition-sensitive occupancy of nucleosomes and/or chromatin macromolecules the method comprising:
(a) comparing, to a reference genome sequence, at least a portion of:
   (i) a plurality of first nucleic acid sequence datasets, each first nucleic acid sequence dataset being obtained from a plurality of digestion-protected regions of a plurality of nucleic acid molecules obtained from a first subject with a first condition, wherein the plurality of first nucleic acid sequence datasets each comprise a plurality of first nucleic acid fragments;
   wherein a genomic location of each of the plurality of first nucleic acid fragments is identified;
(b) comparing, to the reference genome sequence, at least a portion of:
   (i) a plurality of second nucleic acid sequence datasets, each second nucleic acid sequence dataset being obtained from a plurality of digestion-protected regions of a plurality of nucleic acid molecules obtained from a second subject with a second condition, wherein the plurality of second nucleic acid sequence datasets each comprise a plurality of second nucleic acid fragments;
   wherein a genomic location of each of the plurality of second nucleic acid fragments is identified;
(c) (i) determining an average normalised occupancy of digestion-protected regions of the nucleic acid fragments per genomic region (O₁) of the first subjects with the first condition and (ii) determining an average occupancy of digestion-protected regions of nucleic acid fragments per genomic region (O₂) of the second subjects with the second condition; wherein the method optionally comprises repeating step (c) for any other condition N to determine normalised average occupancy of digestion-protected regions of the nucleic acid fragments per genomic region (O_{N}) of the subjects with condition N;
(d) (i) determining one or more stable-nucleosome-occupancy regions of the genome in which the variation of the occupancy of the nucleic acid fragments in digestion-protected regions in each of the first subjects with the first condition is below a first set threshold value; and optionally (d) (ii) determining one or more fuzzy-nucleosome-occupancy regions of the genome in which the variation of the occupancy of the nucleic acid fragments in digestion-protected regions in each of the first subjects with the first condition is above a first set threshold value;
(e) (i) determining one or more stable-nucleosome-occupancy regions of the genome in which the variation of the occupancy of nucleic acid fragments in digestion-protected regions in each of the second subjects with the second condition is below a second set threshold value; and optionally (e) (ii) determining one or more fuzzy-nucleosome-occupancy regions of the genome in which the variation of the occupancy of nucleic acid fragments in digestion-protected regions in each of the second subjects with the second condition is above a second set threshold value;
(f) comparing (i) the one or more stable-nucleosome-occupancy regions or the one or more fuzzy-nucleosome-occupancy regions of the genome of the first subjects with the first condition and (ii) the one or more stable-nucleosome-occupancy regions or the one or more fuzzy-nucleosome-occupancy regions of the genome of the second subjects with the second condition; and
(g) (i) identifying one or more regions of the genome which have stable-nucleosome-occupancy regions in the first condition and stable-nucleosome-occupancy regions in the second condition, and which have a difference between the average occupancy of digestion-protected regions of the nucleic acid fragments in the first condition and the average occupancy of protected regions of nucleic acid fragments in the second condition larger or smaller than the set threshold values, to thereby identify one or more condition-sensitive regions; and/or
(g) (ii) identifying one or more regions of the genome which have stable-nucleosome-occupancy regions in the first condition and fuzzy-nucleosome-occupancy regions in the second condition or fuzzy-nucleosome-occupancy regions in one condition and stable-nucleosome-occupancy regions in another condition, to thereby identify one or more condition-sensitive regions which change their status from stable-nucleosome-occupancy to fuzzy-nucleosome-occupancy between the first and second conditions.

In a further aspect of the present invention there is provided a method for identifying genomic regions with condition-specific protection, which have condition-sensitive occupancy of nucleosomes and/or chromatin macromolecules the method comprising:
(a) comparing, to a reference genome sequence, at least a portion of:
   (i) a plurality of first nucleic acid sequence datasets, each first nucleic acid sequence dataset being obtained from a plurality of digestion-protected regions of a plurality of nucleic acid molecules obtained from a first subject with a first condition, wherein the plurality of first nucleic acid sequence datasets each comprise a plurality of first nucleic acid fragments;
   wherein a genomic location of each of the plurality of first nucleic acid fragments is identified;
(b) comparing, to the reference genome sequence, at least a portion of:
   (i) a plurality of second nucleic acid sequence datasets, each second nucleic acid sequence dataset being obtained from a plurality of digestion-protected regions of a plurality of nucleic acid molecules obtained from a second subject with a second condition, wherein the plurality of second nucleic acid sequence datasets each comprise a plurality of second nucleic acid fragments;
   wherein a genomic location of each of the plurality of second nucleic acid fragments is identified;
(c) (i) determining an average normalised occupancy of digestion-protected regions of the nucleic acid fragments per genomic region (O₁) of the first subjects with the first condition and (ii) determining an average normalised occupancy of digestion-protected regions of nucleic acid fragments per genomic region (O₂) of the second subjects with the second condition; wherein the method optionally comprises repeating step (c) for any other condition N to determine normalised average occupancy of digestion-protected regions of the nucleic acid fragments per genomic region (O_{N}) of the subjects with condition N;
(d) (i) determining one or more stable-nucleosome regions of the genome in which the variation of the occupancy of the nucleic acid fragments in digestion-protected regions in each of the first subjects with the first condition is below a first set threshold value; and optionally (d) (ii) determining one or more fuzzy-nucleosome regions of the genome in which the variation of the occupancy of the nucleic acid fragments in digestion-protected regions in each of the first subjects with the first condition is above a first set threshold value;
(e) (i) determining one or more stable-nucleosome regions of the genome in which the variation of the occupancy of nucleic acid fragments in digestion-protected regions in each of the second subjects with the second condition is below a second set threshold value; and optionally (e) (ii) determining one or more fuzzy-nucleosome regions of the genome in which the variation of the occupancy of nucleic acid fragments in digestion-protected regions in each of the second subjects with the second condition is above a second set threshold value;
(f) comparing (i) the one or more stable-nucleosome regions or the one or more fuzzy-nucleosome regions of the genome of the first subjects with the first condition and (ii) the one or more stable-nucleosome regions or the one or more fuzzy-nucleosomes of the genome of the second subjects with the second condition; and
(g) (i) identifying one or more regions of the genome which have stable-nucleosome regions in the first condition and stable-nucleosome regions in the second condition, and which have a difference between the average occupancy of digestion-protected regions of the nucleic acid fragments in the first condition and the average occupancy of protected regions of nucleic acid fragments in the second condition larger or smaller than the set threshold values, to thereby identify one or more condition-sensitive regions; and/or
(g) (ii) identifying one or more regions of the genome which have stable-nucleosome regions in the first condition and fuzzy-nucleosome regions in the second condition or fuzzy-nucleosome regions in one condition and stable-nucleosome regions in another condition, to thereby identify one or more condition-sensitive regions which change their status from stable-nucleosome region to fuzzy-nucleosome region between the first and second conditions.

In certain embodiments, the stable-nucleosome region is a stable-nucleosome-occupancy region.

In certain embodiments, step (d) comprises (ii) determining one or more fuzzy-nucleosome regions of the genome in which the variation of the occupancy of the nucleic acid fragments in digestion-protected regions in each of the first subjects with the first condition is above a first set threshold value.

In certain embodiments, the fuzzy-nucleosome region is a fuzzy-nucleosome-occupancy region.

In certain embodiments, step (e) comprises (ii) determining one or more fuzzy-nucleosome-occupancy regions of the genome in which the variation of the occupancy of nucleic acid fragments in digestion-protected regions in each of the second subjects with the second condition is above a second set threshold value; and the method further comprises;
(f) comparing (i) the one or more stable-nucleosome-occupancy regions of the genome or the one or more fuzzy-nucleosome-occupancy regions of the first subjects with the first condition and (ii) the one or more stable-nucleosome regions of the genome or the one or more fuzzy-nucleosome-occupancy regions of the second subjects with the second condition; and
(g) (i) identifying one or more regions of the genome which have stable-nucleosome-occupancy regions in the first condition and stable-nucleosome-occupancy regions in the second condition, and which have a difference between the average occupancy of digestion-protected regions of the nucleic acid fragments in the first condition and the average occupancy of protected regions of nucleic acid fragments in the second condition larger or smaller than the set threshold values, to thereby identify one or more condition-sensitive regions; and/or
(g) (ii) identifying one or more regions of the genome which have stable-nucleosome regions in the first condition and fuzzy-nucleosome regions in the second condition or fuzzy-nucleosome regions in one condition and stable-nucleosome regions in another condition, to thereby identify one or more condition-sensitive regions which change their status from stable-nucleosome-occupancy to fuzzy-nucleosome-occupancy between the first and second conditions.

In a further aspect of the present invention, there is provided a method for identifying genomic regions with condition-sensitive positioning of nucleosomes and/or chromatin macromolecules, the method comprising:
(a) comparing, to a reference genome sequence, at least a portion of:
   (i) a plurality of first nucleic acid sequence datasets, each first nucleic acid sequence dataset being obtained from a plurality of digestion-protected regions of a plurality of nucleic acid molecules obtained from a first subject with a first condition, wherein the plurality of first nucleic acid sequence datasets each comprise a plurality of first nucleic acid fragments;
   wherein a genomic location of each of the plurality of first nucleic acid fragments is identified;
(b) comparing, to the reference genome sequence, at least a portion of:
   (i) a plurality of second nucleic acid sequence datasets, each second nucleic acid sequence dataset being obtained from a plurality of digestion-protected regions of a plurality of nucleic acid molecules obtained from a second subject with a second condition, wherein the plurality of second nucleic acid sequence datasets each comprise a plurality of second nucleic acid fragments;
   wherein a genomic location of each of the plurality of second nucleic acid fragments is identified;
(c) (i) determining the genomic locations defined by the region start and region end coordinates of digestion-protected regions of nucleic acid fragments of each of the first subjects with the first condition and (ii) determining the genomic locations of digestion-protected regions of nucleic acid fragments of each of the second subjects with the second condition; wherein the method optionally comprises repeating step (c) for one or more other conditions (N) to determine the genomic locations of digestion-protected regions of nucleic acid fragments of subjects with condition N;
(d) determining one or more stable-nucleosome-positioning regions of the genome in which the variation of the genomic locations defined by the start and end coordinates or the center coordinate of digestion-protected regions of nucleic acid fragments in each of the first subjects with the first condition is below a first set threshold value;
(e) determining one or more stable-nucleosome-positioning regions of the genome in which the variation of the genomic locations defined by the start and end coordinates or the center coordinate of digested-protected regions of nucleic acid fragments in each of the second subjects with the second condition is below a second set threshold value;
(f) comparing (i) the one or more stable-nucleosome-positioning regions of the genome of the first subjects with the first condition and (ii) the one or more stable-nucleosome-positioning regions of the genome of the second subjects with the second condition; and
(g) identifying one or more condition-sensitive regions of the genome which have stable-nucleosome-positioning regions in the first condition and which changed their genomic locations in the second condition by a value larger or smaller than a set threshold value, to thereby identify one or more condition-sensitive genomic regions where the nucleosome locations shifted (“shifted nucleosomes”); and
(h) identifying one or more condition-sensitive regions of the genome which have stable-nucleosome-positioning regions in the first condition and which do not overlap with stable-nucleosome-positioning regions in the second condition, to thereby identify one or more condition-sensitive genomic regions that preferentially contain a nucleosome in the first condition and preferentially lost nucleosome in the second condition (“lost nucleosomes”); (i) identifying one or more condition-sensitive regions of the genome which have stable-nucleosome-positioning regions in the second condition and which do not overlap with stable-nucleosome-positioning regions in the first condition, to thereby identify one or more condition-sensitive genomic regions that preferentially do not contain a nucleosome in the first condition and gained nucleosome in the second condition (“gained nucleosomes”).

In a further aspect of the present invention, there is provided a method for identifying genomic regions with condition-specific protection, which have condition-sensitive occupancy of nucleosomes and/or chromatin macromolecules such as proteins and RNA, the method comprising:
(a) comparing, to a reference genome sequence, at least a portion of:
   (i) a plurality of first nucleic acid sequence datasets, each first nucleic acid sequence dataset being obtained from a plurality of digestion-protected regions of a plurality of nucleic acid molecules obtained from a first subject with a first condition, wherein the plurality of first nucleic acid sequence datasets each comprise a plurality of first nucleic acid fragments;
   wherein a genomic location of each of the plurality of first nucleic acid fragments is identified;
(b) comparing, to the reference genome sequence, at least a portion of:
   (i) a plurality of second nucleic acid sequence datasets, each second nucleic acid sequence dataset being obtained from a plurality of digestion-protected regions of a plurality of nucleic acid molecules obtained from a second subject with a second condition, wherein the plurality of second nucleic acid sequence datasets each comprise a plurality of second nucleic acid fragments;
   wherein a genomic location of each of the plurality of second nucleic acid fragments is identified;
(c) (i) determining an average normalised occupancy of digestion-protected regions of the nucleic acid fragments per genomic region (O₁) of the first subjects with the first condition and (ii) determining an average normalised occupancy of digestion-protected regions of nucleic acid fragments per genomic region (O₂) of the second subjects with the second condition; wherein the method optionally comprises repeating step (c) for any other condition N to determine normalised average normalised occupancy of digestion-protected regions of the nucleic acid fragments per genomic region (O_{N}) of the subjects with condition N;
(d) determining one or more stable-nucleosome regions of the genome in which the variation of the occupancy of the nucleic acid fragments in digestion-protected regions in each of the first subjects with the first condition is below a first set threshold value;
(e) determining one or more stable-nucleosome regions of the genome in which the variation of the occupancy of nucleic acid fragments in digestion-protected regions in each of the second subjects with the second condition is below a second set threshold value;
(f) comparing (i) the one or more stable-nucleosome regions of the genome of the first subjects with the first condition and (ii) the one or more stable-nucleosome regions of the genome of the second subjects with the second condition; and
(g) identifying one or more regions of the genome which have stable-nucleosome regions in the first condition and stable-nucleosome regions in the second condition, and which have a difference between the average occupancy of digestion-protected regions of the nucleic acid fragments in the first condition and the average occupancy of protected regions of nucleic acid fragments in the second condition larger or smaller than the set threshold values, to thereby identify one or more condition-sensitive regions.

In a further aspect of the present invention, there is provided a method for identifying genomic regions with condition-specific protection, which have condition-sensitive occupancy of nucleosomes and/or chromatin macromolecules the method comprising:
(a) comparing, to a reference genome sequence, at least a portion of:
   (i) a plurality of first nucleic acid sequence datasets, each first nucleic acid sequence dataset being obtained from a plurality of digestion-protected regions of a plurality of nucleic acid molecules obtained from a first subject with a first condition, wherein the plurality of first nucleic acid sequence datasets each comprise a plurality of first nucleic acid fragments;
   wherein a genomic location of each of the plurality of first nucleic acid fragments is identified;
(b) comparing, to the reference genome sequence, at least a portion of:
   (i) a plurality of second nucleic acid sequence datasets, each second nucleic acid sequence dataset being obtained from a plurality of digestion-protected regions of a plurality of nucleic acid molecules obtained from a second subject with a second condition, wherein the plurality of second nucleic acid sequence datasets each comprise a plurality of second nucleic acid fragments;
   wherein a genomic location of each of the plurality of second nucleic acid fragments is identified;
(c) (i) determining an average normalised occupancy of digestion-protected regions of the nucleic acid fragments per genomic region (O₁) of the first subjects with the first condition and (ii) determining an average normalised occupancy of digestion-protected regions of nucleic acid fragments per genomic region (O₂) of the second subjects with the second condition; wherein the method optionally comprises repeating step (c) for any other condition N to determine average normalised occupancy of digestion-protected regions of the nucleic acid fragments per genomic region (O_{N}) of the subjects with condition N;
(d) (i) determining one or more stable-nucleosome regions of the genome in which the variation of the occupancy of the nucleic acid fragments in digestion-protected regions in each of the first subjects with the first condition is below a first set threshold value; and
(d) (ii) determining one or more fuzzy-nucleosome regions of the genome in which the variation of the occupancy of the nucleic acid fragments in digestion-protected regions in each of the first subjects with the first condition is above a first set threshold value;
(e) (i) determining one or more stable-nucleosome regions of the genome in which the variation of the occupancy of nucleic acid fragments in digestion-protected regions in each of the second subjects with the second condition is below a second set threshold value; and
(e) (ii) determining one or more fuzzy-nucleosome regions of the genome in which the variation of the occupancy of nucleic acid fragments in digestion-protected regions in each of the second subjects with the second condition is above a second set threshold value;
(f) comparing (i) the one or more stable-nucleosome regions or the one or more fuzzy-nucleosome regions of the genome of the first subjects with the first condition and (ii) the one or more stable-nucleosome regions or the one or more fuzzy-nucleosome regions of the genome of the second subjects with the second condition; and
(g) (i) identifying one or more regions of the genome which have stable-nucleosome regions in the first condition and stable-nucleosome regions in the second condition, and which have a difference between the average occupancy of digestion-protected regions of the nucleic acid fragments in the first condition and the average occupancy of protected regions of nucleic acid fragments in the second condition larger or smaller than the set threshold values, to thereby identify one or more condition-sensitive regions; and/or
(g) (ii) identifying one or more regions of the genome which have stable-nucleosome regions in the first condition and fuzzy-nucleosome regions in the second condition or fuzzy-nucleosome regions in one condition and stable-nucleosome regions in another condition, to thereby identify one or more condition-sensitive regions which change their status from stable-nucleosome region to fuzzy-nucleosome region between the first and second conditions.

In certain embodiments, the chromatin macromolecules may be a protein and/or RNA.

In certain embodiments, the method comprises repeating step (c) for each additional condition (N) to determine average normalised occupancy of digestion-protected regions of the nucleic acid fragments per genomic region (O_{N}) of the subjects with condition N.

In certain embodiments, the method comprises identifying multiple condition-sensitive regions of the genome.

As used herein, the terms “protected region” and “digestion-protected region” refer to a nucleic acid fragment which is protected from digestion by enzymes such as nucleases or chemicals introducing breaks in nucleic acids or from physical factors inducing fragmentation of nucleic acids such as irradiation or sonication. In some embodiments, the protected region is a DNA molecule which is associated with a protein. In some embodiments, the protected region is a DNA molecule which is associated with histone proteins. In some embodiments, the protected region is a DNA molecule wrapped around a histone octamer.

As used herein, the terms “fuzzy nucleosomes”, “fuzzy-nucleosome regions” and fuzzy-nucleosome-occupancy” are used to describe genomic regions that contain varying level of protection from digestion, as judged either by observing different levels of protection of the same region in replicate samples from the same person in the same condition, or by observing different levels of protection of the same region comparing samples from different person with the same condition.

As used herein, the term “stable-nucleosome-positioning” is used to describe DNA fragments protected from DNA digestion, which are well-localized in such a way that the genomic coordinates of the start and end or the center of these DNA fragments do not differ between samples of interest more than a set threshold.

As used herein, the terms “stable-nucleosome” and “stable-nucleosome-occupancy” are used to describe genomic regions where the normalised nucleosome occupancy does not differ between samples of interest more than a set threshold.

As used herein, the terms “condition-sensitive region” and “sensitive-nucleosome region” refer to a region of the genome that contain nucleosomes that are sensitive to a condition.

That is to say an area e.g. a genomic area which differs in a subject with a condition as compared to a subject without the same condition, e.g. in terms of chromatin organisation, nucleosome positioning, nucleosome occupancy, protein binding, cell-free DNA occupancy or cell-free DNA fragment positioning.

In certain embodiments, the method is based on cell-free nucleic acids present in body fluids and/or nucleic acids from living cells.

In certain embodiments, the method further comprises:
(h) identifying one or more regions of the genome which comprise condition-sensitive regions for combinations of different conditions. In certain embodiments, step (h) comprises determining intersections, unions and/or exclusions of condition-sensitive regions, wherein:
   intersections define regions sensitive to each of several conditions of interest,
   unions define regions sensitive to at least one of several conditions of interest and
   exclusions define regions sensitive to some conditions but not sensitive to other specified conditions (for example, sensitive to cancer but not sensitive to ageing).

In certain embodiments, the method further comprises:
(i) refining the set of genomic regions comprising condition-sensitive regions by including or excluding condition-sensitive regions defined for comorbidities. In certain embodiments, the comorbidity may be ageing for example.

In certain embodiments, normalisation of occupancy for a predetermined sample comprises dividing the number of digestion-protected regions of nucleic acid fragments in a predetermined genomic region by the average occupancy for a predetermined genomic region in the predetermined sample in a predetermined condition.

In certain embodiments, normalisation of occupancy for a predetermined sample comprises dividing the number of digestion-protected regions of nucleic acid fragments in a predetermined genomic region by the average occupancy for a larger genomic region enclosing the predetermined genomic region in the predetermined sample in a predetermined condition.

In certain embodiments, normalisation of occupancy for a predetermined sample comprises dividing the number of digestion-protected regions of nucleic acid fragments in a predetermined genomic region by the average occupancy for a genomic region which is established as a reference region with stable (minimally changed) occupancy.

In certain embodiments, step (a) and/or step (b) comprises sequencing the respective nucleic acid fragments, wherein optionally the sequencing comprises single-short-read sequencing, paired-end short-read sequencing and/or or long-read sequencing of nucleic acid fragments along its entire length. The sequencing may be genome-wide or of targeted genomic regions.

In certain embodiments, step (a) and/or step (b) comprises performing paired end sequencing of the respective plurality of nucleic acid fragments to determine unique genomic coordinates of both ends of each nucleic acid fragment.

In certain embodiments, step (c) comprises splitting the reference genomic sequence into regions of a predetermined length and determining average normalised occupancy of protected regions of nucleic acid fragments within each region.

In certain embodiments, the sizes of the genomic regions for the calculation of normalised occupancy are between 10 base pairs (bp) and 100000 bp in length, optionally wherein the regions are 50-150 bp in length. In certain embodiments, the sizes of the genomic regions for the calculation of normalised occupancy are between 10 base pairs (bp) and 10000 bp in length.

In certain embodiments, step (d) comprises applying a first threshold value to identify the one or more stable-nucleosome genomic regions with a variation of the occupancy of digestion-protected regions of nucleic acid fragments across all subjects with the first condition below the first threshold value.

In certain embodiments, step (e) comprises applying a second threshold value to identify the one or more stable-nucleosome genomic regions with a variation of the occupancy of digestion-protected regions of the nucleic acid fragments across all subjects with the second condition below the second threshold value.

In certain embodiments, the method comprises applying a pairwise dissimilarity threshold value defining a minimal acceptable value of the relative difference of an average normalised occupancy of digestion-protected regions of nucleic acid fragments in the first condition (O₁) and nucleic acid fragments in the second condition (O₂), wherein the relative difference is defined as (O₂−O₁)/(O₁+O₂).

In certain embodiments, the method comprises applying a pairwise dissimilarity threshold value defining a minimal acceptable value of the relative difference of the standard deviation of an average normalised occupancy of digestion-protected regions of nucleic acid fragments across all samples in the first condition (Dev(O₁)) and the second condition Dev((O₂)).

In certain embodiments, the condition-sensitive region of the genome comprises a difference between the subjects with the first condition and the subjects with the second condition in one or more of the following, or in a combination of therein:
(i) average profile of the occupancy of protected regions of nucleic acid fragments;
(ii) genomic location of the center of nucleosome;
(iii) genomic locations of the start and end of the nucleosome;
(iv) size of linker DNA between nucleosomes;
(v) stability of nucleosomes against digestion by MNase or another nuclease;
(vi) stability of the nucleosome against partial DNA unwrapping;
(vii) stability of the nucleosome against partial disassembly of the histone octamer;
(viii) accessibility of DNA as measured by ATAC-seq or/and DNase-seq; and/or
(ix) protein binding as measured by ChIP-seq or CUT&RUN or CUT&Tag.

In certain embodiments, the method further comprises, prior to step (a) and/or step (b):
(i) obtaining first nucleic acid sequence data from the digestion-protected regions of the nucleic acid molecules from a plurality of subjects with the first condition, wherein the first nucleic acid sequence data comprises a plurality of first nucleic acid fragments; and/or
(iii) obtaining second nucleic acid sequence data obtained from digestion-protected regions of nucleic acid molecules from a plurality of subjects with the second condition, wherein the second nucleic acid sequence data comprises a plurality of second nucleic acid fragments.

In certain embodiments, which is to identify the target number of condition-sensitive regions, and wherein the method further comprises:
determining a target number of condition-sensitive genomic regions; and/or
altering the predetermined length of the genomic regions; and/or.
altering the threshold values for defining stable-nucleosome regions; and/or
altering the pairwise dissimilarity threshold value.

In certain embodiments the method is to identify the target number of condition-sensitive regions and further comprises:
altering the threshold values for defining stable-nucleosome-positioning regions; and/or
altering the threshold values for defining shifted-nucleosome regions; and/or
altering the threshold values for defining lost-nucleosome regions; and/or
altering the threshold values for defining gained-nucleosome regions.

In certain embodiments, the method further comprises iteratively altering the predetermined length of the genomic regions for the calculation of average occupancy and/or the threshold values for determining stable-nucleosome regions and/or the pairwise dissimilarity threshold value for determining difference between conditions. In certain embodiments, the method comprises iteratively altering the predetermined length of the genomic regions for the calculation of average occupancy and/or the threshold values for determining stable-nucleosome regions and/or the pairwise dissimilarity threshold value for determining difference between conditions two or more times, e.g. 3, 4, 5, 6, 7, 8, 9, or 10 or more times.

In certain embodiments, the method comprises refining condition-sensitive regions of the genome to include a binding site of an overrepresented transcription factor inside condition-sensitive regions. In certain embodiments, the condition-sensitive region(s) comprises a plurality of binding sites of a plurality of overrepresented transcription factors.

In certain embodiments, the method comprises refining condition-sensitive regions of the genome to include or exclude a DNA sequence repeat inside condition-sensitive regions. In certain embodiments, the condition-sensitive region(s) comprises a plurality of DNA sequence repeats.

In certain embodiments, the method is a computer-implemented method.

In certain embodiments, obtaining the plurality of nucleic acid sequence datasets comprises
(i) performing an enzyme digestion of nucleic acid molecules comprised in one or more samples comprising said protected regions of nucleic acid and
(ii) sequencing resultant nucleic acid fragments.

In an embodiment, the enzyme digestion comprises nuclease digestion, for example micrococcal nuclease digestion. In an embodiment, obtaining the one or more nucleic acid sequence datasets comprises (i) probing protected regions of nucleic acid e.g. DNA with a mutant Tn5 transposase to cleave the protected regions of the nucleic acid e.g. DNA and (ii) tagging resultant nucleic acid fragments with one or more sequencing adaptors.

In an embodiment, obtaining the one or more nucleic acid sequence datasets comprises:
(i) chromatin immunoprecipitation; and
(ii) sequencing of immunoprecipitated nucleic acid e.g. DNA fragments; and
(iii) CUT&RUN or CUT&Tag.

In an embodiment, obtaining the one or more nucleic acid sequence datasets comprises performing a technique independently selected from MNase-seq, ATAC-seq, ChIP-seq, CUT&RUN and/or CUT&Tag.

In certain embodiments, the method comprises obtaining cell-free nucleic acids from a sample extracted from at least one of: blood plasma, serum, lymphatic fluid, cerebral spinal fluid, eye humour, urine or other body fluids.

In certain embodiments, the protected regions of DNA are obtained from a sample comprising cell-free DNA.

In certain embodiments, the first condition may be a pathological disorder. The pathological disorder may be for example a cancer, a sub-type of cancer, a viral infection, a bacterial infection, an inflammatory disorder, sepsis, cardiovascular disorder, acute cellular rejection, benign kidney disease, benign liver disease, hepatitis B, inflammatory bowel disease, lupus, diabetes, Crohn's disease, myocarditis, pericarditis, multiple sclerosis, psoriasis and/or a neurological disease. Details of other conditions are provided herein.

In certain embodiments, the first condition is the absence of a pathological disorder.

In certain embodiments, the second condition is a pathological disorder. The pathological disorder may be for example a cancer, a sub-type of cancer, a viral infection, a bacterial infection, an inflammatory disorder, sepsis, cardiovascular disorder, acute cellular rejection, benign kidney disease, benign liver disease, hepatitis B, inflammatory bowel disease, lupus, diabetes, Crohn's disease, myocarditis, pericarditis, multiple sclerosis, psoriasis and/or a neurological disease. Details of other conditions are provided herein.

In certain embodiments, the second condition is the absence of a pathological disorder.

In certain embodiments, the first condition and the second condition are different.

In certain embodiments, the first condition is a pathological disorder and the second condition is the absence of the pathological disorder.

In certain embodiments, the first condition is an age of the subject and the second condition is an age of the subject, wherein the first medical condition and the second medical condition are either the same or different.

In certain embodiments, one of the first and the second condition is different from the respective other condition by the degree of disease progression. In certain embodiments, one of conditions is different from another condition by the degree patient's response to therapy treatment. In certain embodiments, one of conditions is different from another condition by the different time point of obtaining the samples. In certain embodiments, the plurality of first and second subjects are human and wherein the genome is a human genome.

In certain embodiments, one of the first and the second condition is different from the respective other condition by person's lifestyle. In certain embodiments, one of the first and the second condition is different from the respective other condition by person's diet. In certain embodiments, one of the first and the second condition is different from the respective other condition by person's alcohol consumption, smoking and use of other substances.

In a further aspect of the present invention, there is provided a system for identifying condition-sensitive regions in cell-free DNA, the system comprising a computer program configured to;
(a) compare, to a reference genome sequence, at least a portion of:
   (i) a plurality of first nucleic acid sequence datasets, each first nucleic acid sequence dataset being obtained from a plurality of digestion-protected regions of a plurality of nucleic acid molecules obtained from a first subject with a first condition, wherein the plurality of first nucleic acid sequence datasets each comprise a plurality of first nucleic acid fragments;
   wherein a genomic location of each of the plurality of first nucleic acid fragments is identified;
(b) compare, to the reference genome sequence, at least a portion of:
   (i) a plurality of second nucleic acid sequence datasets, each second nucleic acid sequence dataset being obtained from a plurality of digestion-protected regions of a plurality of nucleic acid molecules obtained from a second subject with a second condition, wherein the plurality of second nucleic acid sequence datasets each comprise a plurality of second nucleic acid fragments;
   wherein a genomic location of each of the plurality of second nucleic acid fragments is identified;
(c) (i) determine an average normalised occupancy of digestion-protected regions of nucleic acid fragments per genomic region (O₁) of the first subjects with the first condition and (ii) determine an average normalised occupancy of digestion-protected regions of nucleic acid fragments per genomic region (O₂) of the second subjects with the second condition; optionally repeat step (c) for any other condition N to determine average normalised occupancy of protected regions of nucleic acid fragments per genomic region (O_{N}) of subjects with condition N;
(d) determine one or more stable-nucleosome regions of the genome in which the variation of the occupancy of protected regions of nucleic acid fragments in each of the first subjects with the first condition is below a first set threshold value;
(e) determine one or more stable-nucleosome regions of the genome in which the variation of the occupancy of protected regions of nucleic acid fragments in each of the second subjects with the second condition is below a second set threshold value;
(f) compare (i) the one or more stable-nucleosome regions of the genome of the first subjects with the first condition and (ii) the one or more stable-nucleosome regions of the genome of the second subjects with the second condition; and
(g) identify one or more regions of the genome which have stable-nucleosome regions in the first condition and stable-nucleosome regions in the second condition, and which have the difference between the average occupancy of protected regions of nucleic acid fragments in the first condition and the average occupancy of protected regions of nucleic acid fragments in the second condition larger or smaller than set threshold values, to thereby identify one or more condition-sensitive regions.

In certain embodiments, the stable nucleosome region is a stable-nucleosome-occupancy region.

In certain embodiments, step (d) comprises (ii) determining one or more fuzzy-nucleosome regions of the genome in which the variation of the occupancy of the nucleic acid fragments in digestion-protected regions in each of the first subjects with the first condition is above a first set threshold value.

In certain embodiments, the fuzzy-nucleosome region is a fuzzy-nucleosome-occupancy region.

In certain embodiments, step (e) comprises (ii) determining one or more fuzzy-nucleosome-occupancy regions of the genome in which the variation of the occupancy of nucleic acid fragments in digestion-protected regions in each of the second subjects with the second condition is above a second set threshold value; and the method further comprises;
(f) comparing (i) the one or more stable-nucleosome-occupancy regions of the genome or the one or more fuzzy-nucleosome-occupancy regions of the first subjects with the first condition and (ii) the one or more stable-nucleosome regions of the genome or the one or more fuzzy-nucleosome-occupancy regions of the second subjects with the second condition; and
(g) (i) identifying one or more regions of the genome which have stable-nucleosome-occupancy regions in the first condition and stable-nucleosome-occupancy regions in the second condition, and which have a difference between the average occupancy of digestion-protected regions of the nucleic acid fragments in the first condition and the average occupancy of protected regions of nucleic acid fragments in the second condition larger or smaller than the set threshold values, to thereby identify one or more condition-sensitive regions; and/or
(g) (ii) identifying one or more regions of the genome which have stable-nucleosome regions in the first condition and fuzzy-nucleosome regions in the second condition or fuzzy-nucleosome regions in one condition and stable-nucleosome regions in another condition, to thereby identify one or more condition-sensitive regions which change their status from “stable-nucleosome-occupancy” to “fuzzy-nucleosome-occupancy” between the first and second conditions.

In a further aspect of the present invention, there is provided a system for identifying condition-sensitive regions in cell-free DNA, the system comprising a computer program configured to;
(a) comparing, to a reference genome sequence, at least a portion of:
   (i) a plurality of first nucleic acid sequence datasets, each first nucleic acid sequence dataset being obtained from a plurality of digestion-protected regions of a plurality of nucleic acid molecules obtained from a first subject with a first condition, wherein the plurality of first nucleic acid sequence datasets each comprise a plurality of first nucleic acid fragments;
   wherein a genomic location of each of the plurality of first nucleic acid fragments is identified;
(b) comparing, to the reference genome sequence, at least a portion of:
   (i) a plurality of second nucleic acid sequence datasets, each second nucleic acid sequence dataset being obtained from a plurality of digestion-protected regions of a plurality of nucleic acid molecules obtained from a second subject with a second condition, wherein the plurality of second nucleic acid sequence datasets each comprise a plurality of second nucleic acid fragments;
   wherein a genomic location of each of the plurality of second nucleic acid fragments is identified;
(c) (i) determining the genomic locations defined by the region start and region end coordinates of digestion-protected regions of nucleic acid fragments of each of the first subjects with the first condition and (ii) determining the genomic locations of digestion-protected regions of nucleic acid fragments of each of the second subjects with the second condition; wherein the method optionally comprises repeating step (c) for one or more other conditions (N) to determine the genomic locations of digestion-protected regions of nucleic acid fragments of subjects with condition N;
(d) determining one or more stable-nucleosome-positioning regions of the genome in which the variation of the genomic locations defined by the start and end coordinates or the center coordinates of digestion-protected regions of nucleic acid fragments in each of the first subjects with the first condition is below a first set threshold value;
(e) determining one or more stable-nucleosome-positioning regions of the genome in which the variation of the genomic locations defined by the start and end coordinates or the center coordinates of digested-protected regions of nucleic acid fragments in each of the second subjects with the second condition is below a second set threshold value;
(f) comparing (i) the one or more stable-nucleosome-positioning regions of the genome of the first subjects with the first condition and (ii) the one or more stable-nucleosome-positioning regions of the genome of the second subjects with the second condition; and
(g) identifying one or more condition-sensitive regions of the genome which have stable-nucleosome-positioning regions in the first condition and which changed their genomic locations in the second condition by a value larger or smaller than a set threshold value, to thereby identify one or more condition-sensitive genomic regions where the nucleosome locations shifted (“shifted nucleosomes”); and
(h) identifying one or more condition-sensitive regions of the genome which have stable-nucleosome-positioning regions in the first condition and which do not overlap with stable-nucleosome-positioning regions in the second condition, to thereby identify one or more condition-sensitive genomic regions that preferentially contain a nucleosome in the first condition and preferentially lost nucleosome in the second condition (“lost nucleosomes”); and
(i) identifying one or more condition-sensitive regions of the genome which have stable-nucleosome-positioning regions in the second condition and which do not overlap with stable-nucleosome-positioning regions in the first condition, to thereby identify one or more condition-sensitive genomic regions that preferentially do not contain a nucleosome in the first condition and gained nucleosome in the second condition (“gained nucleosomes”).

In a further aspect of the present invention there is provided a system for identifying genomic regions which have condition-sensitive occupancy of nucleosomes and/or chromatin macromolecules the system configured to:
(a) compare, to a reference genome sequence, at least a portion of:
   (i) a plurality of first nucleic acid sequence datasets, each first nucleic acid sequence dataset being obtained from a plurality of digestion-protected regions of a plurality of nucleic acid molecules obtained from a first subject with a first condition, wherein the plurality of first nucleic acid sequence datasets each comprise a plurality of first nucleic acid fragments;
   wherein a genomic location of each of the plurality of first nucleic acid fragments is identified;
(b) compare, to the reference genome sequence, at least a portion of:
   (i) a plurality of second nucleic acid sequence datasets, each second nucleic acid sequence dataset being obtained from a plurality of digestion-protected regions of a plurality of nucleic acid molecules obtained from a second subject with a second condition, wherein the plurality of second nucleic acid sequence datasets each comprise a plurality of second nucleic acid fragments;
   wherein a genomic location of each of the plurality of second nucleic acid fragments is identified;
(c) (i) determine an average normalised occupancy of digestion-protected regions of the nucleic acid fragments per genomic region (O₁) of the first subjects with the first condition and (ii) determine an average normalised occupancy of digestion-protected regions of nucleic acid fragments per genomic region (O₂) of the second subjects with the second condition; wherein the system is optionally configured to repeat step (c) for any other condition N to determine average normalised occupancy of digestion-protected regions of the nucleic acid fragments per genomic region (O_{N}) of the subjects with condition N;
(d) (i) determine one or more stable-nucleosome regions of the genome in which the variation of the occupancy of the nucleic acid fragments in digestion-protected regions in each of the first subjects with the first condition is below a first set threshold value; and optionally (d) (ii) determine one or more fuzzy-nucleosome regions of the genome in which the variation of the occupancy of the nucleic acid fragments in digestion-protected regions in each of the first subjects with the first condition is above a first set threshold value;
(e) (i) determine one or more stable-nucleosome regions of the genome in which the variation of the occupancy of nucleic acid fragments in digestion-protected regions in each of the second subjects with the second condition is below a second set threshold value; and optionally (e) (ii) determine one or more fuzzy-nucleosome regions of the genome in which the variation of the occupancy of nucleic acid fragments in digestion-protected regions in each of the second subjects with the second condition is above a second set threshold value;
(f) compare (i) the one or more stable-nucleosome regions or the one or more fuzzy-nucleosome regions of the genome of the first subjects with the first condition and (ii) the one or more stable-nucleosome regions or the one or more fuzzy-nucleosome regions of the genome of the second subjects with the second condition; and
(g) (i) identify one or more regions of the genome which have stable-nucleosome regions in the first condition and stable-nucleosome regions in the second condition, and which have a difference between the average occupancy of digestion-protected regions of the nucleic acid fragments in the first condition and the average occupancy of protected regions of nucleic acid fragments in the second condition larger or smaller than the set threshold values, to thereby identify one or more condition-sensitive regions; and/or
(g) (ii) identify one or more regions of the genome which have stable-nucleosome regions in the first condition and fuzzy-nucleosome regions in the second condition or fuzzy-nucleosome regions in one condition and stable-nucleosome regions in another condition, to thereby identify one or more condition-sensitive regions which change their status from stable-nucleosome region to fuzzy nucleosome region between the first and second conditions.

In a further aspect of the present invention, there is provided a system for identifying condition-sensitive regions in cell-free DNA, the system comprising a computer program configured:
(a) compare, to a reference genome sequence, at least a portion of:
   (i) a plurality of first nucleic acid sequence datasets, each first nucleic acid sequence dataset being obtained from a plurality of digestion-protected regions of a plurality of nucleic acid molecules obtained from a first subject with a first condition, wherein the plurality of first nucleic acid sequence datasets each comprise a plurality of first nucleic acid fragments;
   wherein a genomic location of each of the plurality of first nucleic acid fragments is identified;
(b) compare, to the reference genome sequence, at least a portion of:
   (i) a plurality of second nucleic acid sequence datasets, each second nucleic acid sequence dataset being obtained from a plurality of digestion-protected regions of a plurality of nucleic acid molecules obtained from a second subject with a second condition, wherein the plurality of second nucleic acid sequence datasets each comprise a plurality of second nucleic acid fragments;
   wherein a genomic location of each of the plurality of second nucleic acid fragments is identified;
(c) (i) determine an average normalised occupancy of digestion-protected regions of the nucleic acid fragments per genomic region (O₁) of the first subjects with the first condition and (ii) determine an average normalised occupancy of digestion-protected regions of nucleic acid fragments per genomic region (O₂) of the second subjects with the second condition; wherein optionally the program is configured to repeat step (c) for any other condition N to determine average normalised occupancy of digestion-protected regions of the nucleic acid fragments per genomic region (O_{N}) of the subjects with condition N;
(d) (i) determine one or more stable-nucleosome regions of the genome in which the variation of the occupancy of the nucleic acid fragments in digestion-protected regions in each of the first subjects with the first condition is below a first set threshold value; and
(d) (ii) determine one or more fuzzy-nucleosome regions of the genome in which the variation of the occupancy of the nucleic acid fragments in digestion-protected regions in each of the first subjects with the first condition is above a first set threshold value;
(e) (i) determine one or more stable-nucleosome regions of the genome in which the variation of the occupancy of nucleic acid fragments in digestion-protected regions in each of the second subjects with the second condition is below a second set threshold value; and
(e) (ii) determine one or more fuzzy-nucleosome regions of the genome in which the variation of the occupancy of nucleic acid fragments in digestion-protected regions in each of the second subjects with the second condition is above a second set threshold value;
(f) compare (i) the one or more stable-nucleosome regions or the one or more fuzzy-nucleosome regions of the genome of the first subjects with the first condition and (ii) the one or more stable-nucleosome regions or the one or more fuzzy-nucleosome regions of the genome of the second subjects with the second condition; and
(g) (i) identify one or more regions of the genome which have stable-nucleosome regions in the first condition and stable-nucleosome regions in the second condition, and which have a difference between the average occupancy of digestion-protected regions of the nucleic acid fragments in the first condition and the average occupancy of protected regions of nucleic acid fragments in the second condition larger or smaller than the set threshold values, to thereby identify one or more condition-sensitive regions; and/or
(g) (ii) identify one or more regions of the genome which have stable-nucleosome regions in the first condition and fuzzy-nucleosome regions in the second condition or fuzzy-nucleosome regions in one condition and stable-nucleosome regions in another condition, to thereby identify one or more condition-sensitive regions which change their status from stable-nucleosome region to fuzzy nucleosome region between the first and second conditions.

In certain embodiments, the chromatin macromolecules may be a protein and/or RNA.

In certain embodiments, the system is further configured to;
(h) identify one or more regions of the genome which comprise condition-sensitive regions for combinations of different conditions by determining intersections, unions and/or exclusions of condition-sensitive regions, wherein:
   intersections define regions sensitive to each of several conditions of interest),
   unions are composed of condition-sensitive regions defined for more than two pairs of conditions of interest. Unions thus define regions sensitive to at least one of several conditions of interest and
   exclusions define regions sensitive to some conditions but not sensitive to one or more other conditions (for example, sensitive to cancer but not sensitive to ageing); and
(i) refine the set of condition-sensitive regions by including or excluding condition-sensitive regions defined for one or more comorbidities; and/or
(j) refine the set of condition-sensitive regions by including or excluding DNA sequence repeats or transcription factor binding sites overlapping with these regions.

In an embodiment, the comorbidity is aging.

In certain embodiments, the system is further configured to:
refine the set of genomic regions comprising condition-sensitive regions by including or excluding condition-sensitive regions defined for comorbidities. In certain embodiments, the comorbidity may be ageing for example.

In certain embodiments, the system is configured to normalise occupancy for a predetermined sample by dividing the number of digestion-protected regions of nucleic acid fragments in a predetermined genomic region by the average occupancy for a predetermined genomic region in a predetermined sample in a predetermined condition.

In certain embodiments, the normalisation of occupancy for a predetermined sample comprises dividing the number of digestion-protected genomic regions of nucleic acid fragments in a predetermined genomic region by the average occupancy for a larger genomic region enclosing the predetermined genomic region in a predetermined sample in a predetermined condition.

In certain embodiments, normalisation of occupancy for a predetermined sample comprises dividing the number of digestion-protected regions of nucleic acid fragments in a predetermined genomic region by the average occupancy for a genomic region which is established as a reference region with stable (minimally changed) occupancy.

In certain embodiments, the system is configured to sequencing the respective nucleic acid fragments, wherein optionally the sequencing comprises single-short-read sequencing, paired-end short-read sequencing and/or or long-read sequencing of nucleic acid fragments along its entire length. The sequencing may be genome-wide or of targeted genomic regions.

In certain embodiments, the system is configured to perform paired end sequencing of the respective plurality of nucleic acid fragments to determine unique genomic coordinates of both ends of each nucleic acid fragment.

In certain embodiments, the system is configured to split the reference genomic sequence into regions of a predetermined length and determining average normalised occupancy of protected regions of nucleic acid fragments within each region.

In certain embodiments, the sizes of the genomic regions for the calculation of normalised occupancy are between 10 base pairs (bp) and 100000 bp in length, optionally wherein the regions are 50-150 bp in length. In certain embodiments, the sizes of the genomic regions for the calculation of normalised occupancy are between 10 base pairs (bp) and 100000 bp in length.

In certain embodiments, the system is configured to apply a first threshold value to identify the one or more stable-nucleosome genomic regions with a variation of the occupancy of digestion-protected regions of nucleic acid fragments across all subjects with the first condition below the first threshold value.

In certain embodiments, the system is configured to apply a second threshold value to identify the one or more stable-nucleosome genomic regions with a variation of the occupancy of digestion-protected regions of the nucleic acid fragments across all subjects with the second condition below the second threshold value.

In certain embodiments, the system is configured to apply a pairwise dissimilarity threshold value defining a minimal acceptable value of the relative difference of an average normalised occupancy of digestion-protected regions of nucleic acid fragments in the first condition (O₁) and nucleic acid fragments in the second condition (O₂), wherein the relative difference is defined as (O₂−O₁)/(01+O₂).

In certain embodiments, the system is configured to apply a pairwise dissimilarity threshold value defining a minimal acceptable value of the relative difference of the standard deviation of an average normalised occupancy of digestion-protected regions of nucleic acid fragments across all samples in the first condition (Dev(O₁)) and the second condition Dev((O₂)).

The systems of certain aspects of the present invention may include one or more components such as a computer, software, algorithms and hardware.

In a further aspect of the present invention there is provided a method of identifying a condition in a subject, the method comprising:
(a) defining one or more characteristics for a set of condition-sensitive regions of a genome;
(b) defining a set of condition-sensitive regions by performing a method for identifying genomic regions which have condition-sensitive occupancy of nucleosomes and/or chromatin macromolecules as described herein;
(c) obtaining nucleic acid sequence data from at least a portion of cell free DNA (cfDNA) isolated from a sample from the subject, wherein the subject is a first subject in which a condition is to be determined;
(d) performing an alignment of the nucleic acid sequence data to the reference genome to define the genomic coordinates of sequenced reads;
(e) calculating a normalised occupancy of cfDNA per genomic region separately for each sample;
(f) creating a reference set of samples, each of which are known to be obtained from a subject having a predetermined condition;
(g) calculating an average normalised occupancy of cfDNA, separately for each sample in the reference set for each condition-specific region;
(h) performing dimensionality reduction analysis on (i) the sample obtained from the first subject in which the condition needs to be determined and (ii) the samples from the reference set of samples; and
(j) performing a classification of the sample from the first subject based on the similarity of the average normalised cfDNA occupancy in condition-sensitive regions to clusters formed by the samples from the reference set.

In certain embodiments, the classification is a multiple-conditions classification.

In certain embodiments, a characteristic of step (a) comprises the condition-sensitive region comprising a binding site of an overrepresented transcription factor. In certain embodiments, the condition-sensitive region(s) comprises a plurality of binding sites of a plurality of overrepresented transcription factors.

In certain embodiments, a characteristic of step (a) comprises refining condition-sensitive regions of the genome to include or exclude a DNA sequence repeat inside condition-sensitive regions. In certain embodiments, the condition-sensitive region(s) comprises a plurality of DNA sequence repeats.

In certain embodiments, the normalisation is performed by dividing the number of digestion-protected regions of nucleic acid fragments in a predetermined region by the average occupancy for a predetermined chromosome in a predetermined sample in a predetermined condition.

In certain embodiments, the normalisation is performed by dividing the number of digestion-protected regions of nucleic acid fragments in a predetermined region by an average occupancy for a larger region enclosed a predetermined region on a predetermined genomic location in a predetermined sample in a predetermined condition.

In certain embodiments, normalisation of occupancy for a predetermined sample comprises dividing the number of digestion-protected regions of nucleic acid fragments in a predetermined genomic region by the average occupancy for a genomic region which is established as a reference region with stable (minimally changed) occupancy.

In certain embodiments, the reference set of samples comprises around 3-6 samples per condition.

In certain embodiments, the dimensionality reduction analysis comprises principal component analysis (PCA).

In some embodiments, the method comprises identifying a genomic coordinate of a nucleic acid fragment on the chromosome. As used herein the genomic coordinate is the number defining the location of a fragment on the chromosome in a genome assembly. In some embodiments, the method comprises identifying the type of DNA sequence repeats whose location cannot be mapped exactly on the chromosome.

In certain embodiments, the method is for identifying condition-sensitive regions of the genome of a plurality of subjects. In certain embodiments, the subjects are human subjects.

In certain embodiments, sample classification is performed based on condition-sensitive region by machine learning, linear regression, logistic regression, support vector machines (SVM), convolutional neural networks (CNN) and/or deep learning.

### BRIEF DESCRIPTION OF DRAWINGS

Certain embodiments of the present invention will now be described hereinafter, by way of example only, with reference to the accompanying drawings in which:

FIG. 1 shows a diagram depicting that circulating cell-free DNA (cfDNA) in body fluids comes from processes such as apoptosis, necrosis or NETosis, where DNA nucleases cut genomic DNA preferentially between nucleosomes. In the healthy person, most cfDNA in blood plasma has been released from blood cells. In patients with a disease the fraction of cfDNA originating from the diseased cell types may increase.

FIG. 2 shows application of cfDNA nucleosomics analysis to distinguish between three medical conditions, breast cancer, liver cancer and lupus using data from [Snyder et al., (2016) Cell 164, 57-58]. A) PCA performed using nucleosome occupancy values in all gene promoters. B) PCA performed using nucleosome occupancy values in “sensitive-nucleosome regions” defined by using cfDNA from healthy people and breast cancer patients as detailed in the current invention. Note that cfDNA from healthy controls and breast cancer patients was used to define the sensitive regions, but cfDNA from patients with lupus and liver cancer was not used for the definition of sensitive nucleosome regions, but nevertheless our method is able to diagnose these medical conditions not used for model training.

FIG. 3 shows the effect of ageing on the sizes of cfDNA fragments (A) and on the patterns of nucleosome occupancy in age-sensitive genomic regions (B). Experimental data from [Teo et al (2019), Aging Cell, 18, e12890]. Panel B shows that PCA analysis based on sensitive-nucleosome regions distinguished person's age.

FIG. 4 is a chart outlining a method according to certain embodiments of the present invention.

FIG. 5 shows application of cfDNA nucleosomics analysis to distinguish between healthy and breast cancer samples from [Snyder et al., (2016) Cell 164, 57-58]. PCA is performed using nucleosome occupancy values in “lost-nucleosome regions” defined by using cfDNA from healthy people and breast cancer patients as detailed herein.

### DETAILED DESCRIPTION

Further features of certain embodiments of the present invention are described below. The practice of embodiments of the present invention will employ, unless otherwise indicated, conventional techniques of molecular biology, microbiology, recombinant DNA technology and immunology, which are within the skill of those working in the art.

Most general molecular biology, microbiology recombinant DNA technology and immunological techniques can be found in Sambrook et al, Molecular Cloning, A Laboratory Manual (2001) Cold Harbor-Laboratory Press, Cold Spring Harbor, N.Y. or Ausubel et al., Current protocols in molecular biology (1990) John Wiley and Sons, N.Y. Unless defined otherwise, all technical and scientific terms used herein have the same meaning as commonly understood by one of ordinary skill in the art to which this disclosure belongs. For example, the Concise Dictionary of Biomedicine and Molecular Biology, Juo, Pei-Show, 2nd ed., 2002, CRC Press; The Dictionary of Cell and Molecular Biology, 3rd ed., Academic Press; and the Oxford University Press, provide a person skilled in the art with a general dictionary of many of the terms used in this disclosure.

Units, prefixes and symbols are denoted in their Système International de Unitese (SI) accepted form. Numeric ranges are inclusive of the numbers defining the range.

Aspects of the present invention provide a method to define condition-sensitive regions. Aptly, the method may be used to define condition-sensitive genomic regions present in the cfDNA of liquid biopsies of a subject. Aptly, assessment of the identified condition-sensitive genomic regions may form part of a liquid biopsy assay, which may then be used to diagnose, monitor a treatment response, and/or stratify a patient.

The term “subject” “as used herein may refer to any animal, mammal, or human. In some embodiments, the subject is a human.

Aptly, the methods described herein may identify regions in a genome which are stable-nucleosome regions. The genome may be a human genome.

The term “genomic region” as used herein generally refers to any region of the genome (e.g., a range of base pair positions), e.g., the entire genome, chromosome, gene, or exon. The genomic region may be a continuous or discontinuous region. A “locus” (or “locus”) can be part or all of a genomic region (e.g., part of a gene, or a single nucleotide of a gene).

The methods and system of certain embodiments comprise the use of a “reference genome”. The term “reference genome” is used to refer to a nucleic acid sequence database that is assembled from genetic data and intended to represent the genome of a species. Aptly, the reference genome is haploid. Aptly, the reference genome does not represent the genome of a single individual of that species, but rather is a mosaic of several individual genomes.

A reference human genome may be hg19. The hg19 human genome is disclosed https://www.ncbi.nim.a nih.gov/assembly/GCF_000001405.13/.

In alternative embodiments, the reference human genome is GRCh38.p13 nov/assembly/GCF_000001405.39

As used herein the term “liquid biopsy” refers to the sampling and analysis of non-solid biological tissue. This is a powerful diagnostic and monitoring tool and has the benefit of being largely non-invasive, and so can be carried out more frequently. Non-limiting examples of liquid biopsy' sources include blood, saliva, sputum, urine or other bodily fluids. The predominant source of liquid biopsies is blood. Liquid biopsies may be collected and purified by any means known in the art, with the method of extraction likely to depend on the source of the biopsy and the desired application.

A wide variety of biomarkers may be sampled and studied from the collected liquid biopsy, to detect or monitor a range of diseases and/or conditions. Aptly, the type of biomarker sampled from the liquid biopsy is dependent on the condition being tested and/or diagnosed. For example if the condition is cancer, then circulating tumor cells (CTCs) and/or circulating tumor DNA (ctDNA) are collected, whereas if the condition is a myocardial infarction, circulating endothelial cells (CECs) are sampled.

As used herein the term “cell-free DNA” and “circulating cell-free DNA (cfDNA)” refers to non-encapsulated DNA (deoxyribonucleic acid) in the liquid biopsy. These nucleic acid fragments are usually of varying size, with over-representation of sizes similar to the length of DNA wrapped around a histone octamer, as well as its multiples. A nucleosome is the combination of DNA wrapped around the histone octamer. The length of the protected DNA within each nucleosome is about 147 base pairs. The protein core of each nucleosome consists of a histone octamer with a subunit stoichiometry of (H2A-H2B)-(H3-H4)-(H3-H4)-(H2A-H2B). A 147 bp segment of DNA is wrapped around the histone octamer in 1.65 turns. Together, the histone octamer and DNA wrapped around it constitute the nucleosome core particle. Histone H1 (linker histone) is also involved in nucleosome packing and is likely to be responsible for control of gene.

Although the mechanisms of cfDNA release are not entirely understood, it is known that cfDNA can enter the bloodstream (or other bodily fluids) as a result of apoptosis or necrosis, as well as active extraction of sections of nucleic acids from the cell (e.g. in NETosis). Elevated cfDNA levels correlate with all-causes mortality and so cfDNA is generally considered as a prognostic factor and a biomarker. Based on the characteristics and accessibility of cfDNA it is deemed a biomarker of growing interest as a tool in diagnostics and therapy efficiency monitoring.

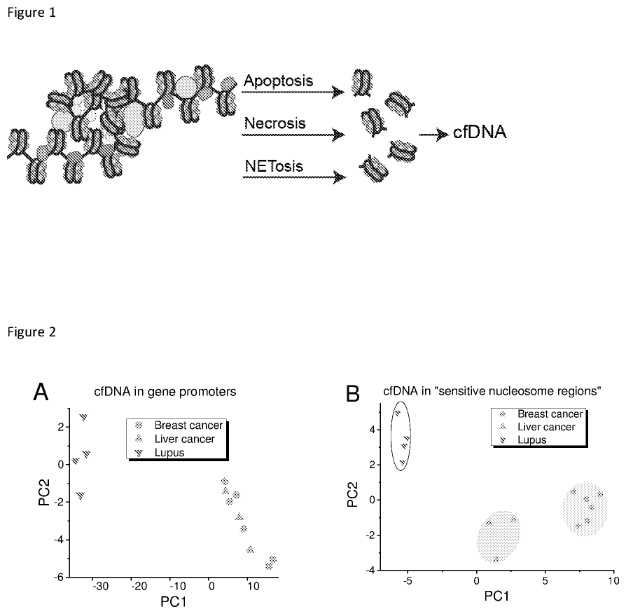

Aptly a liquid biopsy may comprise one or more sub-types of cfDNA including, but not limited to, circulating tumor DNA (ctDNA), circulating cell-free mitochondrial DNA (ccf mtDNA), and cell-free fetal DNA (cffDNA). cfDNA may be collected and purified by any means known in the art, with the method of extraction likely to depend on source of liquid biopsy and the desired application. As shown in FIG. 1, circulating cell-free DNA (cfDNA) in body fluids comes from processes such as apoptosis, necrosis or NETosis, where DNA nucleases cut genomic DNA preferentially between nucleosomes. In the healthy person, cfDNA in blood plasma has been released from blood cells as well as a smaller fraction from other cell types. In patients with a disease the fraction of cfDNA originating from the diseased cell types may increase. In healthy people the amount of cfDNA can differ depending on their physical activity, stress, environmental conditions and other aspect of the life cycle.

Certain embodiments of the present invention comprise sequencing one or more regions of a nucleic acid molecule. In certain embodiments, the nucleic acid molecule is a protein-associated DNA molecule e.g. a DNA molecule which is wrapped around a histone octamer.

In certain embodiments, information regarding the protein-wrapped DNA molecule is provided in a database e.g. a database comprising details of cell-free DNA from a plurality of subjects.

In certain embodiments, sequencing of protein-wrapped DNA e.g. cfDNA is based on published cfDNA datasets. An example of a database comprising cfDNA datasets is NucPosDB (https://generegulation.org/cfdina). NucPosDB also comprises nucleosome positioning maps in vivo (https://generegulation.org/nucposdb/).

In certain embodiments, the method comprises identifying nucleic acid molecules that are comprised in a sample comprising cfDNA. Optionally, the sample is obtained from a subject with a condition. The nucleic acid molecules may be processed to provide a plurality of reads. In one instance, these read-outs may include determining changes of nucleosome occupancy. In one instance, changes of nucleosome occupancy derived from cfDNA may be compared with nucleosome occupancy in normal/disease tissues for tissues involved in a predefined condition, using methods such as MNase-seq, ATAC-seq, ChIP-seq or related.

MNase-seq (micrococcal nuclease digestion with deep sequencing) is a technique used to measure DNA protection by nucleosomes. The technique relies upon the non-specific endo-exonuclease micrococcal nuclease, an enzyme derived from Staphylococcus aureus to bind and cleave protein-unbound regions of DNA on chromatin. DNA bound to histones or other chromatin-bound proteins is preferentially protected from digestion. The uncut DNA is then purified and sequenced.

In certain embodiments, MNase-seq may be combined with or substituted by ATAC-seq, CUT&RUN and/or CUT&Tag sequencing.

CUT&RUN sequencing, which is also known as cleavage under targets and release using nuclease, is a technique combining antibody-targeted controlled cleavage by micrococcal nuclease with massively parallel sequencing.

CUT&Tag sequencing (Cleavage under Targets and Tagmentation) is based on ChIP principles i.e. antibody-based binding of the target protein or histone modification of interest but instead of an immunoprecipitation step, antibody incubation is directly followed by shearing of the chromatin and library preparation.

In certain embodiments, the method comprises obtaining nucleic acid sequence information using an ATAC-seq (Assay for Transposase-Accessible Chromatin using sequencing) technique. ATAC-seq utilises hyperactive transposases to insert transposable markers with specific adapters, capable of binding primers for sequencing, into open regions of chromatin. Sequences adjacent to the inserted transposons can be amplified allowing for determination of accessibly chromatin regions.

In certain embodiments, the method comprises obtaining nucleic acid sequence information using a ChIP-seq (chromatin immunoprecipitation followed by sequencing) technique. Typically the ChIP method uses an antibody for a specific DNA-binding protein or a histone modification to identify enriched loci within a genome. ChIP-seq can be performed on live cells as well as on circulating nucleosomes or fragments of cfDNA bound to proteins while released to body fluids.

Isolated cfDNA may be analysed by any means known in the art, non-limiting examples include 1^{st} generation sequencing techniques such as Maxam-Gilbert sequencing and Sanger sequencing; next generation sequencing techniques such as pyrosequencing (Roche 454); sequencing by ligation (SOLID); sequencing by synthesis (Illumina); IonTorrent/Ion Proton (ThermoFisher); long-read sequencing including SMRT sequencing (Pacific Biosciences) and

Nanopore sequencing (Oxford Nanopore); polymerase chain reaction (PCR), PCR amplicon sequencing, hybrid capture sequencing, enzyme-linked immunosorbent assays (ELISA) and other methods. As a non-limiting example, cfDNA may be analysed by PCR to assess a specific nucleotide sequence, alternatively the cfDNA may be analysed by DNA sequencing methods to assess all the cfDNA present in the sample. Suitable DNA sequencing methods include, but are not limited to, PCR amplicon sequencing, hybrid capture sequencing, or any method known in the art. As a further non-limiting example, isolated cfDNA may be analysed by massively parallel sequencing (MPS). In particular, any appropriate method should aptly avoid contamination, especially in relation to ruptured blood cells.

Next-generation sequencing method which may have utility in embodiments of the present invention include for example massive parallel sequencing. NGS platforms include Roche 454, Illumina NextSeq, Illumina MiSeq, Illumina HiSeq, Illumina Genome Analyser IIX, Life Technologies SOLID, Pacific Biosciences SMRT, ThermoFisher Ion Torrent/Ion Proton, Oxford Nanopore MinION, Oxford Nanopore GridION and Oxford Nanopore PromethION.

In certain embodiments, the methods and system comprise identifying a nucleosome position of a nucleic acid sequence.

As used herein the term nucleosome positioning refers to the location of nucleosomes with respect to the genomic DNA sequence. The nucleosome is the basic unit of eukaryotic chromatin, consisting of a histone core around which DNA is wrapped. Each nucleosome typically contains 147 base pairs (bp) of DNA, which is wrapped around the histone octamer.

The location of nucleosomes along the DNA and their chemical and compositional modifications are key to gene expression—and concomitant cell regulation. Thus, genomic nucleosome positions are non-random and reflect the unique biological processes of each cell. Compared to the slow changes reflected in DNA mutations or aberrant methylation-which may accumulate relatively slowly-genomic nucleosome positions provide almost real-time information on cell function and disease state. Thus, information on nucleosome positioning can provide a valuable diagnostic marker. However, obtaining genome-wide nucleosome positioning maps based on tissues involved in disease, for example tumour tissues of cancer patients, is an expensive and invasive procedure. On the other hand, inferring nucleosome positioning from cfDNA is less invasive.

Without being bound by theory, cfDNA is generated by nucleases, which shred the chromatin of cells including cells undergoing apoptosis, necrosis or NETosis, these enzymes preferentially cut the DNA between nucleosomes. Therefore, nucleosome positioning is reflected in the cfDNA fragmentation patterns. Moreover, since the half-life of cfDNA in blood is in the range of several minutes, cfDNA extracted at any given time point represents a very recent snapshot of nucleosome positioning in the cells of origin.

In certain embodiments, the method and system comprise determining occupancy of the nucleosome in an individual sample and/or an average nucleosome occupancy of a predetermined cohort of subjects. For example, certain embodiments comprise determining an average nucleosome occupancy of a set of subjects having the same condition.

Positioning and occupancy of nucleosomes are closely related concepts; nucleosome positioning is the distribution of individual nucleosomes along the DNA sequence and can be thought of in terms of a single reference point on the nucleosome, such as its center (dyad). Nucleosome occupancy, on the other hand, is a measure of the probability that a certain DNA region is wrapped onto a histone octamer.

As used herein and as described above the terms “condition-sensitive regions”, “condition-sensitive genomic regions” and “sensitive-nucleosome regions”, refer to regions where DNA protection changes in a condition-specific manner. Nucleosome positioning and/or DNA-protein binding in these regions undergoes changes characteristic to a given condition; such changes being an analytical characteristic that can also inform about the severity of condition. Thus, not only can such condition-sensitive regions be used to distinguish between healthy and non-healthy subjects, but also between different medical conditions, between different levels of severity of the same medical condition and between different conditions of a healthy person. Differences in the regions may be as a result of different process such as NETosis employing a different combination of enzymes, thus DNA fragments may have differing nucleotide profiles in subjects with differing conditions. Alternatively or in addition, the condition sensitive regions may differ in size distribution between conditions. In certain embodiments, the difference may be GC content as a function of the distance from the end of a cfDNA fragment.

In certain embodiments, the condition-sensitive region may comprise a binding site of an overrepresented transcription factor. The transcription factor may be for example CTCF, BRD4, RBPJ, SOX2, POU3F2, OLIG2, ARNT2, ASCL1 or MYC. In certain embodiments, a subject with a first condition comprises a condition-sensitive region comprising a greater number of transcription factor sites as compared to the corresponding region in a normal subject i.e. a subject who is not suffering from a pathological disorder.

In certain embodiments, the condition-sensitive region may comprise a DNA sequence repeat. Depending on the experimental sequencing procedure, the dataset of condition-sensitive regions can be refined to include or exclude DNA sequence repeats.

Certain embodiments of the present invention provide a method of selecting condition-sensitive regions. Aptly the condition-sensitive regions are present in cfDNA.

Aptly the condition-sensitive genomic regions are capable of distinguishing between different medical conditions, including but not limited to, different types of cancer and systemic inflammation, as well as the problem of determining biological age in healthy individuals. Consequently, the applicability of condition-sensitive regions as part of liquid biopsy clinical tools is general.

In certain embodiments, the method and systems comprise determining regions of a genome which are substantially the same within a subject class e.g. subjects which each have a condition. In certain embodiments, the method comprises obtaining a read from a cfDNA sample e.g. a cfDNA sample comprised in a dataset.

The term “read” refers to a sequence read from a portion of a nucleic acid sample. Typically, though not necessarily, a read represents a short sequence of contiguous base pairs in the sample. The read may be represented symbolically by the base pair sequence (in ATCG) of the sample portion. It may be stored in a memory device and processed as appropriate to determine whether it matches a reference sequence or meets other criteria. A read may be obtained directly from a sequencing apparatus or indirectly from stored sequence information concerning the sample. In some cases, a read is a DNA sequence of sufficient length (e.g., at least about 10 bp) that can be used to identify a larger sequence or region, e.g. that can be aligned to the reference genome and specifically assigned to a chromosome or an extra-chromosomal location inside the cell.

In certain embodiments, the method comprises the use of threshold values. As used herein the term “threshold” refers to a predetermined number used in an operation. For example, a threshold value can refer to a value above or below which a particular classification applies.

In certain embodiments, the first condition and/or the second condition may be a cancer. In certain embodiments, the first and/or second condition is a subtype of a cancer.

In certain embodiments, the subject has a malignant tumour. The cancer may be selected from the group consisting of: solid tumours such as melanoma, skin cancers, small cell lung cancer, non-small cell lung cancer, glioma, hepatocellular (liver) carcinoma, gallbladder cancer, thyroid tumour, bone cancer, gastric (stomach) cancer, prostate cancer, breast cancer, ovarian cancer, cervical cancer, uterine cancer, vulval cancer, endometrial cancer, testicular cancer, bladder cancer, lung cancer, glioblastoma, endometrial cancer, kidney cancer, renal cell carcinoma, colon cancer, colorectal, pancreatic cancer, oesophageal carcinoma, brain/CNS cancers, head and neck cancers, neuronal cancers, mesothelioma, sarcomas, biliary (cholangiocarcinoma), small bowel adenocarcinoma, paediatric malignancies, epidermoid carcinoma, sarcomas, cancer of the pleural/peritoneal membranes and leukaemia, including acute myeloid leukaemia, acute lymphoblastic leukaemia, and multiple myeloma.

In certain embodiments, the condition may be a neoplastic disease, for example, melanoma, skin cancer, small cell lung cancer, non-small cell lung cancer, salivary gland, glioma, hepatocellular (liver) carcinoma, gallbladder cancer, thyroid tumour, bone cancer, gastric (stomach) cancer, prostate cancer, breast cancer, ovarian cancer, cervical cancer, uterine cancer, vulval cancer, endometrial cancer, testicular cancer, bladder cancer, lung cancer, glioblastoma, thyroid cancer, endometrial cancer, kidney cancer, colon cancer, colorectal cancer, pancreatic cancer, oesophageal carcinoma, brain/CNS cancers, neuronal cancers, head and neck cancers, mesothelioma, sarcomas, biliary (cholangiocarcinoma), small bowel adenocarcinoma, paediatric malignancies, epidermoid carcinoma, sarcomas, cancer of the pleural/peritoneal membranes and leukaemia, including acute myeloid leukaemia, acute lymphoblastic leukaemia, and multiple myeloma. Treatable chronic viral infections include HIV, hepatitis B virus (HBV), and hepatitis C virus (HCV) in humans, simian immunodeficiency virus (SIV) in monkeys, and lymphocytic choriomeningitis virus (LCMV) in mice.

In certain embodiments, the condition may comprise disease-related cell invasion and/or proliferation. Disease-related cell invasion and/or proliferation may be any abnormal, undesirable or pathological cell invasion and/or proliferation, for example tumour-related cell invasion and/or proliferation.

In one embodiment, the neoplastic disease is a solid tumour selected from any one of the following carcinomas of the breast, colon, colorectal, prostate, stomach, gastric, ovary, oesophagus, pancreas, gallbladder, non-small cell lung cancer, thyroid, endometrium, head and neck, renal, renal cell carcinoma, bladder and gliomas.

In certain embodiments, the first and/or second condition may comprise a subtype of a condition. For example in certain embodiments, the first condition may be a subtype of a cancer and the second condition may be a further subtype of a cancer. By way of example only, the first condition may be a biomarker-positive cancer e.g. HER2+ breast cancer and the second condition may be a biomarker-negative cancer e.g. HER2 negative breast cancer.

In certain embodiments, the first condition may be a predetermined age e.g. a predetermined age range and the second condition is a further predetermined age e.g. a further predetermined age range which differs from the first age range.

In certain embodiments, the first and/or second condition is an inflammatory disorder. The inflammatory disorder may be selected from lupus, asthma, rheumatoid arthritis, ulcerative colitis, Crohn's disease, myocarditis, pericarditis, multiple sclerosis, psoriasis and the like.

In certain embodiments, the first and/or second condition is an autoimmune disorder.

In certain embodiments, the first condition is a pathological disorder and the second condition is absence of a pathological disorder e.g. the subject with the first condition is a subject suffering from a pathological disorder and the subject with the second condition is a healthy subject. In certain embodiments, the subject with the first condition is a subject suffering from a pathological disorder and the subject with the second condition is a subject suffering from a different pathological disorder to the subject with the first condition.

In some embodiments, the method comprises comparing the subject with the first condition or the subject with the second condition is a reference subject. In certain embodiments, the reference subject is healthy. In some embodiments, the reference subject has a disease or disorder, optionally selected from the group consisting of: cancer, normal pregnancy, a complication of pregnancy (e.g., aneuploid pregnancy), myocardial infarction, inflammatory bowel disease, systemic autoimmune disease, localized autoimmune disease, allotransplantation with rejection, allotransplantation without rejection, stroke, and localized tissue damage.

In certain embodiments, one of the first and the second condition is different from the respective other condition by person's lifestyle. In certain embodiments, one of the first and the second condition is different from the respective other condition by person's diet. In certain embodiments, one of the first and the second condition is different from the respective other condition by person's alcohol consumption, smoking and use of other substances.

### Methods of Diagnosing a Condition

In certain embodiments, the method comprises defining the optimal requirements and characteristics for the set of condition-specific genomic regions based on the required level of diagnostic confidence and the available budget and scale of operation which may affect the number of genomic regions analysed and also based on the employed experimental sequencing technique, which may affect the sizes of the regions. In an embodiment, the method comprises a step of refining the set of condition-specific genomic regions which comprises selecting regions which comprise a binding site of a transcription factor that is overrepresented in a condition. The transcription factor may be for example CTCF, BRD4, RBPJ, SOX2, POU3F2, OLIG2, ARNT2, ASCL1 or MYC. In certain embodiments, a subject with a first condition comprises a condition-sensitive region comprising a greater number of transcription factor sites as compared to the corresponding region in a normal subject i.e. a subject who is not suffering from a pathological disorder. In an embodiment, the method comprises a step of refining the set of condition-specific genomic regions which comprises including or excluding regions which overlap with a DNA sequence repeat.

The present disclosure also provides methods of diagnosing a disease or disorder using condition-sensitive regions identified by the method according to the present invention and as disclosed herein.

In certain embodiments, the regions selected as detailed herein are then used for comparison of nucleosome occupancy across samples, which can be done with a number of computational approaches.

In one embodiment, the method comprises the use of dimensionality reduction techniques, such as principal component analysis (PCA) as in the example in FIG. 2. In certain embodiments, the method comprises the use of other dimensionality reduction techniques such as t-distributed stochastic neighbour embedding (tSNE), k-means clustering, or unsupervised clustering. In certain embodiments, the method comprises of the use of machine learning techniques such as linear regression, logistic regression, support vector machines (SVM) and/or convolutional neural networks (CNN).

In the example shown in FIG. 2, three different medical conditions: breast cancer, liver cancer and lupus (systemic inflammation) are distinguished. FIG. 2A shows PCA analysis based on the comparison of nucleosome occupancy at gene promoter regions. As it is clear from this figure, while lupus can be distinguished from cancer using this method, two cancer types (breast cancer and liver cancer) cannot be distinguished from each other. On the other hand, FIG. 2B shows PCA analysis based on the regions harbouring “sensitive-nucleosomes” defined by the method of certain embodiments. In the latter case all three medical conditions can be clearly separated. This demonstrates that the method according to certain embodiments of the present invention is significantly more efficient than previous methods.

As described herein, the condition-sensitive regions may be identified from cell free DNA obtained from subjects having a known disorder or disease or defined clinical condition ((e.g. normal, pregnancy, cancer type A, cancer type B, etc.))

In certain embodiments, the method comprises obtaining a sample comprising cell-free DNA from a subject suspected of having or having a condition.

Thus, in certain embodiments, the method comprises use of Principal Component Analysis (PCA). As used herein principal component analysis (PCA) is a technique for reducing the dimensionality of datasets. In order to interpret large datasets, methods are required that drastically reduce the dataset's dimensionality in an interpretable manner, while also preserving the information in the data. PCA is an adaptive descriptive data analysis tool, which creates new uncorrelated variables that successively maximize variance. This methodology reduces a dataset's dimensionality, thereby increasing interpretability but at the same time minimizing information loss. Furthermore, PCA can be effectively tailored to various data types and structures, hence can be used in numerous situations and disciplines.

In certain embodiments, the method comprises identifying at least six condition-sensitive regions in a subject having or suspected of having a condition. In certain embodiments, the method comprises identifying at least ten condition-sensitive regions in a subject having or suspected of having a condition. It will be appreciated that the method may comprise identifying more than ten condition-sensitive regions e.g. 11, 12, 13, 14, 15, 16, 17, 18, 19, 20 or more.

In certain embodiments, the method comprises performing one or more analysis e.g. classification/clustering/machine learning analysis.

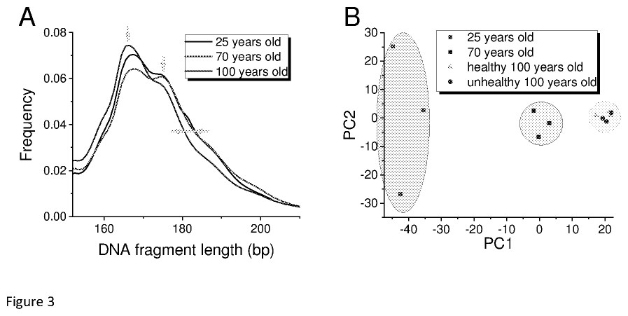

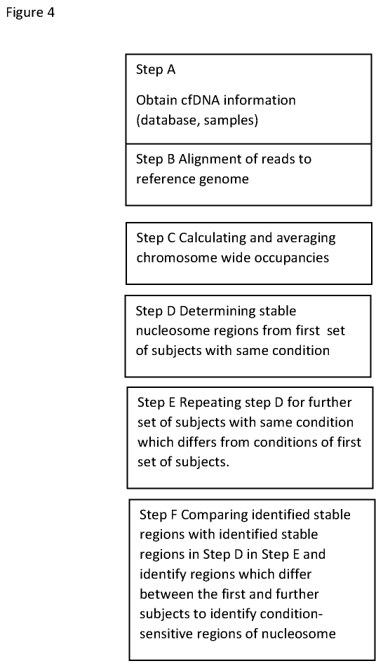

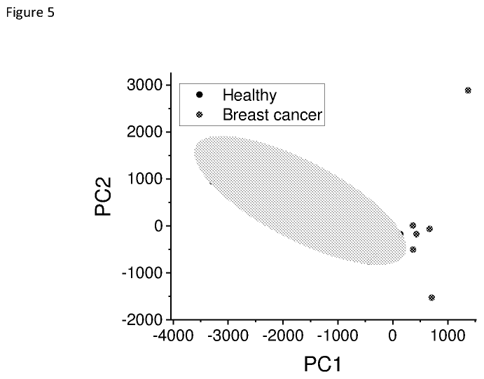

In certain embodiments, the method comprises exclusion of one or more co-morbidities. Particularly, in certain embodiments, the method allows fine-tuning sensitive genomic regions to include/exclude the effect of different comorbidities. For example, one of the most common problems is that cancer patients of different age have different cfDNA patterns. It is important to distinguish healthy ageing from different medical conditions. The inventors have identified a new effect of cfDNA shortening in old people (FIG. 3A) and have compiled a set of age-sensitive genomic regions that can be used for the estimation of the patient's age based on cfDNA (FIG. 3B). Selecting cancer-sensitive regions (C1) that do not overlap with age-sensitive regions (C2) can improve the robustness of cancer diagnostics, because cancer patients of different age have both cancer-specific cfDNA changes and age-specific cfDNA changes. Excluding age-specific cfDNA changes allows to focus only on cancer-specific cfDNA changes. Similarly, the method of certain embodiments allows excluding other comorbidities-sensitive regions from sets of regions used in cfDNA-based medical diagnostics.

In certain embodiments, condition-specific changes of nucleosome positioning may include for example condition-specific changes of the average profiles of the occupancy of nucleosomes, the locations of centers of nucleosomes, the sizes of the linker DNA between nucleosomes, the stability of nucleosomes against MNase digestion, the stability of the nucleosome against partial DNA unwrapping, the stability of the nucleosome against partial disassembly of the histone octamer, the accessibility of DNA inside nucleosomes to protein binding, as well as any related changes affecting the nucleosome landscape.

In certain embodiments of the present invention, a system is provided which is configured to perform the methods of the invention. Aptly, the system is a computer-implemented system. The computer system can control various aspects of the disclosed method. The computer system may include a central processing unit (CPU), also referred to as a processor or computer processor. In certain embodiments, the processor may be a plurality of processors. The computer system may communicate with a memory or memory location. The computer system may comprise a computer or a mobile computer device e.g. a smartphone or a tablet. Also included in the computer system may be an electronic storage unit and one or more other systems.

Computer storage includes for example random access memory (RAM), read only memory (ROM), or any other medium capable of storing computer-readable instructions. The computer may include or have access to a computing environment that includes an input, an output and a communication connection. The input may include one or more of a touchscreen, touchpad, mouse, keyboard, camera, one or more device-specific buttons and other input devices. Computer-readable instructions stored on a computer-readable medium may be executable by a processing unit of the computer. Examples of non-transitory computer-readable mediums include a hard drive (magnetic disk or solid state), CD-ROM and RAM. The system may also comprise software, hardware, algorithms and/or workflows to implement the methods of certain embodiments of the present invention.

The methods and systems of the present disclosure can be implemented by one or more algorithms. The algorithm can be implemented by software when executed by a processor. In certain embodiments, determining the condition-sensitive regions may comprise the use of software packages, Nuctools (https://generegulation.org/nuctools), BedTools (https://bedtools.readthedocs.io/en/latest/), Bowtie or Bowtie2 (http://bowtie-bio.sourceforge.net/index. shtml), as well as other general-purpose bioinformatics tools for next generation sequencing analysis and custom-made scripts.

Nuctools is also described in
Vainshtein, Y., Rippe, K. & Teif, V. B. “NucTools: analysis of chromatin feature occupancy profiles from high-throughput sequencing data.” BMC Genomics 18, 158 (2017). https://doi.org/10.1186/s12864-017-3580-2.

BedTools is also described in
Quinlan A R, Hall I M. 2010. “BEDTools: a flexible suite of utilities for comparing genomic features.” Bioinformatics 26:841-842. https://doi.org/10.1093/bioinformatics/btq033

Bowtie is also described in
Langmead B, Trapnell C, Pop M, Salzberg S L. “Ultrafast and memory-efficient alignment of short DNA sequences to the human genome.” Genome Biol 10: R25. https://doi.org/10.1186/gb-2009 Oct. 3-r25

The following is an example of determining condition-specific regions for the case where two conditions used to determine condition-sensitive regions refer to healthy people from two age groups, 25 years old (condition 1) and 100 years old (condition 2). Two additional groups were not used in the initial definition of age-specific regions, but used later to show that the age-specific regions determined based on conditions 1 and 2 allow also to distinguish other age groups. A third group comprised of healthy 70 years old people (condition 3) and fourth group comprised of 100 years old people with some underlying medical issues (condition 4). Steps 1-8 below provide details of the implementation of this analysis.

Step 1. Download raw sequencing data reported in [Teo Y V, Capri M, Morsiani C, Pizza G et al. Cell-free DNA as a biomarker of aging. Aging Cell 2019 February; 18 (1): e12890. PMID: 30575273] described in the GEO entry GSE114511 stored in SRA archive (https://www.ncbi.nlm.nih.gov/sra?term=SRP147273), which includes three samples for condition 1, three samples for condition 2 and three samples for condition 3. Download from SRA archive can be performed using command fastqdump from the SRA Toolkit software package (https://github.com/ncbi/sra-tools).

Step 2. Align paired-end reads downloaded at the previous step using Bowtie, then create individual directories for each sample, use NucTools to convert the aligned reads file from Bowtie's output MAP format for a BED format (paired reads on two consecutive lines), followed by a conversion of this BED format to the BED format with one line per paired read (columns as follows: chromosome, start of fragment, end of fragment, length of fragment), then split this file into individual chromosomes, as detailed in the shell script below:

for i in SRR*
do
cd /example/GSE114511_cfDNA_Teo/${i}
# mapping paired-end reads with Bowtie
bowtie -t -v 2 -p 8 -m 1 --solexa-quals hg19 -1 ${i}_1.fastq.gz -2 ${i}_2.fastq.gz ${i}.map
# Converting aligned reads file fom MAP to BED format
perl NucTools/bowtie2bed.pl ${i}.map ${i}.bed
# Converting BED file from one line per sequenced read to one line per DNA fragment
perl NucTools/extend_PE_reads.pl --input ${i}.bed --output ${i}_nucleosomes.bed
# Split the BED file containing all maped reads per sample into one file per chromosome:
perl NucTools/extract_chr_bed.pl --input=${i}_nucleosomes.bed --pattern=all
done

Step 3. Create individual directories per each chromosome and calculate normalised cfDNA occupancies per sample with a sliding window 100 bp. The shell script below shows an example for Condition 1 (25 years old people). This step needs to be repeated for all conditions.

mkdir chr1
mkdir chr2
mkdir chr3
mkdir chr4
mkdir chr5
mkdir chr6
mkdir chr7
mkdir chr8
mkdir chr9
mkdir chr10
mkdir chr11
mkdir chr12
mkdir chr13
mkdir chr14
mkdir chr15
mkdir chr16
mkdir chr17
mkdir chr18
mkdir chr19
mkdir chr20
mkdir chr21
mkdir chr22
mkdir chrX
mkdir chrY
for j in 7170698 7170699 7170700
do
for i in 1 2 3 4 5 6 7 8 9 10 11 12 13 14 15 16 17 18 19 20 21 22 X Y
do
perl /NucTools/bed2occupancy_average.pl --
input=/example/GSE114511_cfDNA_Teo/SRR${j}/chr${i}.bed --
outdir=/example/Teo_100bp/Teo_25yrs_old_100bp/chr${i} --
output=chr${i}_SRR${i}_100bp.occ --window=100
done
done

Step 4. Using NucTools script stable_nucs_replicates.pl, determine a set of stable-nucleosome regions where the variation of cfDNA occupancy in different samples within the same condition is below a threshold value. The threshold value (−StableThreshold) is selected as 0.5 for both conditions in the example below (under Step 5). For each stable-nucleosome region, this script will calculate the value of the variation and the averaged nucleosome occupancy per condition.

Step 5. Compare stable-nucleosome regions in condition 1 and condition 2 using NucTools script compare_two_conditions.pl to determine regions where the relative change of cfDNA occupancy is below threshold1 (−0.95 in this example) or above threshold2 (0.95 in this example). The output files contain coordinates of condition-sensitive regions where cfDNA occupancy in 100-years old increases in comparison with 25-years old (containing in file titles “100yo_more_25yo”) or decreases (containing in file titles “100yo_more_25yo”). These files are output by default split into chromosomes and can be merged at a later stage to include all chromosomes together. In the example shell script below steps 4 and 5 are combined.

```
for i in 1 2 3 4 5 6 7 8 9 10 11 12 13 14 15 16 17 18 19 20 21 22 X Y
do
perl NucTools/stable_nucs_replicates.pl --fileExtention=occ --
inputDir=/example/Teo_100bp/Teo_25yrs_old_100bp/chr${i} --
outputS=chr${i}_average_25yo_Stable_100bp.txt \
  --coordsCol=0 --occupCol=1 --StableThreshold=0.5 --chromosome=“chr$i” --window=100
perl NucTools/stable_nucs_replicates.pl --fileExtention=occ --
inputDir=/example/Teo_100bp/Teo_100yrs_old_100bp/chr${i} --
outputS=chr${i}_average_100yo_Stable_100bp.txt \
  --coordsCol=0 --occupCol=1 --StableThreshold=0.5 --chromosome=“chr$i” --window=100
perl NucTools/compare_two_conditions.pl --
input1=/example/Teo_100bp/Compare_100yo_vs_25yo/chr${i}_average_25yo_Stable_100b
p.txt --
input2=/example/Teo_100bp/Compare_100yo_vs_25yo/chr${i}_average_100yo_Stable_100
bp.txt \
  --output1=chr${i}_100yo_less_25yo_0.95.txt --output2=chr${i}_100yo_more_25yo_0.95.txt
--chromosome=“chr$i” --windowSize=100 --threshold1=0.95 --threshold2 =-0.95 --
Col_coord=1 --Col_signal=3 --Col_StDev=4 --Col_RelErr=5
done
```

Step 6. Select genomic regions defined at the previous step (either those where cfDNA occupancy increases in condition 2 vs 1 or where it decreases in condition 2 vs 1 or a combination of these), prepare it in BED file format, and use this BED file to create a matrix with cfDNA occupancies in each of these regions for each sample in each condition. To do so, use BedTools to intersect sequentially the BED file containing condition-sensitive regions with the BED files containing stable-nucleosome regions for each sample in each condition. In the example below, we perform this analysis for age-sensitive regions where cfDNA occupancy decreases in 100 years old people in comparison with 25 year old people. The use of BedTools command “intersectbed” with parameter-wo allows to add columns from all samples that are intersected. The shell script below demonstrates this analysis:

# prepare the first intersection:
bedtools intersect -a chr1_SRR7170698_100bp_corrected.bed -b
chr1_100yo_less_25yo_0.95.txt -u > 100yo_less_25yo_chr1_98_100bp.bed
# intersect with other 25yo samples:
bedtools intersect -a 100yo_less_25yo_chr1_98_100bp.bed -b
chr1_SRR7170699_100bp_corrected.bed -wo > 100yo_less_25yo_chr1_98_99_100bp.bed
bedtools intersect -a 100yo_less_25yo_chr1_98_99_100bp.bed -b
chr1_SRR7170700_100bp_corrected.bed -wo > 100yo_less_25yo_chr1_25yo_100bp.bed
# intersect with 70yo samples:
bedtools intersect -a 100yo_less_25yo_chr1_25yo_100bp.bed -b
chr1_SRR7170701_100bp_corrected.bed -wo >
100yo_less_25yo_chr1_25yo_01_100bp.bed
bedtools intersect -a 100yo_less_25yo_chr1_25yo_01_100bp.bed -b
chr1_SRR7170702_100bp_corrected.bed -wo >
100yo_less_25yo_chr1_25yo_01_02_100bp.bed
bedtools intersect -a 100yo_less_25yo_chr1_25yo_01_02_100bp.bed -b
chr1_SRR7170703_100bp_corrected.bed -wo >
100yo_less_25yo_chr1_25yo_70yo_100bp.bed
# intersect with 100yo samples:
bedtools intersect -a 100yo_less_25yo_chr1_25yo_70yo_100bp.bed -b
chr1_SRR7170704_100bp_corrected.bed -wo >
100yo_less_25yo_chr1_25yo_70yo_04_100bp.bed
bedtools intersect -a 100yo_less_25yo_chr1_25yo_70yo_04_100bp.bed -b
chr1_SRR7170705_100bp_corrected.bed -wo >
100yo_less_25yo_chr1_25yo_70yo_04_05_100bp.bed
bedtools intersect -a 100yo_less_25yo_chr1_25yo_70yo_04_05_100bp.bed -b
chr1_SRR7170706_100bp_corrected.bed -wo >
100yo_less_25yo_chr1_25yo_70yo_04_05_06_100bp.bed
bedtools intersect -a 100yo_less_25yo_chr1_25yo_70yo_04_05_06_100bp.bed -b
chr1_SRR7170707_100bp_corrected.bed -wo >
100yo_less_25yo_chr1_25yo_70yo_04_05_06_07_100bp.bed
bedtools intersect -a 100yo_less_25yo_chr1_25yo_70yo_04_05_06_07_100bp.bed -b
chr1_SRR7170708_100bp_corrected.bed -wo >
100yo_less_25yo_chr1_25yo_70yo_04_05_06_07_08_100bp.bed
bedtools intersect -a 100yo_less_25yo_chr1_25yo_70yo_04_05_06_07_08_100bp.bed -b
chr1_SRR7170709_100bp_corrected.bed -wo >
100yo_less_25yo_chr1_25yo_70yo_100yo_100bp.bed

Step 7. Format the resulting file to remove genomic coordinates and keep only the matrix of normalised cfDNA occupancies. Then use this matrix to perform principal component analysis (PCA) using a custom R script demonstrated below:

setwd(“Example_path/Teo_PCA”)
data.ageing <-
read.table(“Example_path/Teo_PCA/100yo_less_25yo_chr1_25yo_70yo_100yo_100bp.bed
”)
head(data.ageing, n=10)
data.ageing<-data.ageing[,c(4,8,13,18,23,28,33,38,43,48,53,58)]
colnames(data.ageing)<- c(“25F”, “25F”, “25M”, “70F”, “70F”, “70M”, “100HF”, “100HF”,
“100HM”, “100UF”, “100UF”,”100UF”)
data.ageing<-t(data.ageing)
n <- ncol(data.ageing)
colnames(data.ageing) <- c(1:n)
data.ageing.pca <- prcomp(data.ageing, center=TRUE, scale=TRUE)
data.ageing.group <- c(rep(“25 year olds”, 3), rep(“70 year olds”, 3), rep(“healthy 100 year
olds”, 3), rep(“unhealthy 100 year olds”, 3))
pca.ageing <- data.ageing.pca$x
write.csv(pca.ageing, “Teo_PCA.csv”)

Step 8. The results of the PCA analysis can be visualised e.g. as in FIG. 3B to demonstrate clustering of different conditions (three clusters for three age groups in this example).

### EXAMPLES

In the following, the invention will be explained in more detail by means of non-limiting examples of specific embodiments.

### Calculations Setup.

Calculations shown in FIGS. 2 and 3 above were performed using the University of Essex computational cluster, ceres.essex.ac.uk. Software packages NucTools [1], BedTools [Quinlan A R, Hall I M. 2010. “BEDTools: a flexible suite of utilities for comparing genomic features.” Bioinformatics 26:841-842] and Bowtie [Langmead B, Trapnell C, Pop M, Salzberg S L. “Ultrafast and memory-efficient alignment of short DNA sequences to the human genome.” Genome Biol 10: R25] and complementary R and Shell scripts included herein were used to perform data processing. The calculation of the histogram of cfDNA fragment size distribution and principal component analysis were performed in R. OriginPro 2020 (originlab.com) was used for graphing.

### Downloading Data.

Fastq files with raw reads from the aforementioned studies were obtained from the Short Read Archive (SRA) (accession numbers SRR212994-SRR2129120 for Snyder et al [2] and SRR7170698-SRR7170709 for Teo et al [3]) using SRA Tools to download the files from SRA and split files into two as the original libraries are paired-end in both studies.

### Reads Alignment and Pre-Processing.

The sequencing reads were mapped to the hg19 human reference genome using Bowtie [4] with parameters set for paired-end reads, allowing up to 2 mismatches, only considering uniquely mappable reads, suppressing all alignments for a read if more than 1 reportable alignments exist for it. The following pre-processing was performed with NucTools. The output Bowtie.map files were converted to BED format using bowtie2bed.pl script (part of NucTools package), and the paired-end reads were combined into one line, adding the fragment length as a new column using NucTools script extend_PE_reads.pl. The mapped.bed files were split into individual chromosomes using NucTools script “extract_chr_bed.pl”.

Calculation of cfDNA Fragment Size Distribution.

The histogram of DNA fragment size distribution was calculated using an R script, “make_hist_from_fraglengths.r” (see below), which takes.bed files with nucleosomes generated by NucTools as input and produces histograms with fragment sizes in.txt format. These were then visualised in Origin (originlab.com).

### Calculating and Averaging Chromosome-Wide Occupancies.

The nucleosome occupancy profiles for individual samples were calculated using NucTools script “bed2occupancy_average.pl”, taking aligned reads in.bed files as an input and producing.occ files for each chromosome with occupancy calculated within 100 bp windows.

Determining Stable-Nucleosome-Occupancy Regions within One Condition.

To determine the locations of the “stable” regions where nucleosome occupancy does not change more than the set threshold for all samples within a given condition, we used the NucTools script “stable_nucs_replicates.pl”. For the example calculations shown in FIGS. 2 and 3, we choose the threshold for relative error between datasets to be less than 0.5 for “stable” nucleosome occupancy regions. Stable nucleosome occupancies were calculated as described above for each of the two conditions used in the comparison. For example, in all breast cancer samples, and separately in all healthy samples from Snyder et al. for the calculation of FIG. 2. In another example, for all 100-year-old people and separately in all 25-year-old people from the Teo et al dataset for the calculation of FIG. 3.

### Comparison of Nucleosome Occupancy Between Conditions.

Stable nucleosome occupancies defined as explained above were compared using the NucTools script “stable_nucs_replicates.pl”. This script takes two files for each compared condition from the previous step and produces.txt files with information on gained or lost occupancy. For both calculations, a window size of 100 bp was chosen (−window=100), so the genome was split into 100 bp regions and the occupancy within each region was averaged. The threshold for relative occupancy change between the averaged occupancies in each condition in “compare_two_conditions.pl” was set for 0.95. As a result of this comparison, two separate datasets were obtained for the genomic regions that lost and gained nucleosomes in one condition in comparison with the other condition.

### Intersecting Genomic Regions for the Nucleosome Occupancy Analysis.

The “bedtools intersect” command was used to find intersecting regions between the datasets with normalised nucleosome occupancies and the files containing condition-sensitive genomic regions. Specifically for the calculation shown in FIG. 2, the genomic regions that had decreased cfDNA occupancy in breast cancer vs normal were intersected with the NucTools-generated files for the cfDNA occupancies in stable regions for each of the samples in all conditions used in the multi-classification analysis. This generated a matrix with rows corresponding to regions that lost nucleosomes in breast cancer, and columns corresponding to the average nucleosome occupancy values for a given 100-bp window in each of the analysed patients and healthy individuals. Similarly, for the calculation shown in FIG. 3, the regions that lost nucleosome occupancy in 100-years old people vs 25-years olds were used for the intersections.

### Principal Component Analysis.

The matrix of nucleosome occupancies in condition-sensitive regions obtained at the previous step was transposed and used for the principal component analysis (PCA) as follows. The condition-sensitive regions were used for PCA based on the values of average nucleosome occupancies in regions that lost nucleosomes in breast cancer compared to healthy for FIG. 2 or in 100-year old people compared to 25-year-olds for FIG. 3. The same workflow for PCA was repeated by intersecting with promoters instead of lost or gained occupancy files for the sake of comparison. PCA was performed in R and plotted in Origin. The R codes are detailed below.

R Script to Calculate a Histogram of cfDNA Fragment Sizes:

args = commandArgs(trailingOnly=TRUE);
file_in=args[1]
file_out=args[2]
library(readr) #you may need to install this with ‘install.packages(‘readr’)’
nucs=read_delim(file_in, delim=“\t”, col_names=F)
colnames(nucs)=c(“chr”, “start”, “end”, “frag_length”)
h=hist(nucs$frag_length, breaks=200, plot=F) #change the number of bins with the ‘breaks'
parameter
dataoi=cbind(h$breaks, c(h$counts, NA), c(h$density, NA))
colnames(dataoi)=c(“Breaks”, “Counts”, “Density”)
write.table(dataoi, file_out, sep=“\t”, row.names=F) #writes the histogram data to a text file
which you can then plot in origin
png(“histogram.png”)
plot(dataoi[,1],dataoi[,2],type=‘I’,xlab=‘frag_lengths',ylab=‘Frequency’)
dev.off( )

R Script to Calculate PCA (in this Case for Ageing Data from Teo et al Based on Nucleosome Occupancies at Promoters):

setwd(“Example_path/Teo_PCA”)
data.ageing <-
read.table(“Example_path/Teo_PCA/100yo_less_25yo_chr1_25yo_70yo_100yo_100bp.bed
”)
head(data.ageing, n=10)
data.ageing<-data.ageing[,c(4,8, 13, 18,23,28,33,38,43,48,53,58)]
colnames(data.ageing)<- c(“25F”, “25F”, “25M”, “70F”, “70F”, “70M”, “100HF”, “100HF”,
“100HM”, “100UF”, “100UF”,”100UF”)
data.ageing<-t(data.ageing)
n <- ncol(data.ageing)
colnames(data.ageing) <- c(1:n)
data.ageing.pca <- prcomp(data.ageing, center=TRUE, scale=TRUE)
data.ageing.group <- c(rep(“25 year olds”, 3), rep(“70 year olds”, 3), rep(“healthy 100 year
olds”, 3), rep(“unhealthy 100 year olds”, 3))
pca.ageing <- data.ageing.pca$x
write.csv(pca.ageing, “Teo_PCA.csv”)

### Defining “Shifted”, “Lost” and “Gained” Nucleosomes.

A method to define condition-sensitive regions is based on locations where an individual nucleosome is well-positioned across subjects with condition 1 but not in condition 2. For example, FIG. 5 shows results of the following calculation. First, cell-free DNA dataset from Snyder et al [2] was used to define nucleosomes that are lost in breast cancer patients versus healthy controls. Then these condition-sensitive regions were used for PCA based on cfDNA occupancy as detailed above. The procedure of defining nucleosomes lost in breast cancer involves the following steps:
1) Define stable nucleosomes in healthy samples as cfDNA fragments whose start and end genomic coordinates do not change more than 1% across all subjects with a given condition. For the calculation in FIG. 5, this was performed by intersecting NucTools-formatted BED files with all mapped cfDNA fragments with sizes between 120-180 bp from chromosome 1 across 4 healthy cfDNA samples, using BEDTools command “intersect” requiring minimal overlap 99% (parameters-u-f 0.99).
2) Define stable nucleosomes in breast cancer samples as cfDNA fragments whose start and end genomic coordinates do not change more than 1% across all subjects with a given condition. For the calculation in FIG. 5, this was performed by intersecting NucTools-formatted BED files with all mapped cfDNA fragments with sizes between 120-180 bp from chromosome 1 across 6 healthy cfDNA samples, using BEDTools command “intersect” requiring minimal overlap 99% (parameters-u-f 0.99).
3) Intersect BED file containing stable nucleosomes in healthy controls obtained on step (1) with BED file containing stable nucleosomes in breast cancer obtained on step (2), using BEDTools command “intersect” with parameter “−v” (which means report only regions of the first dataset that do not have any overlapping with regions in the second dataset). As a result a BED file was obtained with genomic locations of all nucleosomes on chromosome 1 that have stable positioning in healthy controls but do not overlap with stably positioned nucleosomes in breast cancer (denoted as “lost” nucleosomes) (BEDTools intersect parameter “−v”).

The set of nucleosomes lost in breast cancer obtained by steps (1-3) was used to perform PCA analysis based on cfDNA occupancy as detailed above. The results of the PCA analysis are shown in FIG. 5.

In a similar way, it is possible to define “gained” nucleosomes (nucleosomes “gained” in breast cancer), where step (3) is modified to report only stable nucleosomes in breast cancer that do not overlap with stable nucleosomes in healthy.

In a similar way, it is possible to define “shifted” nucleosomes (nucleosomes shifted in breast cancer in comparison with locations of stable nucleosomes in healthy samples). This can be achieved by modifying step (3) above to report only nucleosomes whose locations shifted more than a set threshold. For example, to define nucleosomes whose locations shifted >20%, BEDTools command “intersect” needs to be run with parameters-f 0.80-r-v.

Throughout the description and claims of this specification, the words “comprise” and “contain” and variations of them mean “including but not limited to” and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any foregoing embodiments. The invention extends to any novel one, or novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### REFERENCES

1 Volik et al. Mol Cancer Res 14, 898-908 (2016).
2 Peng et al. Briefings in Bioinformatics (2020).
3 Wan, J. C. M. et al. Liquid biopsies come of age: towards implementation of circulating tumour DNA. Nat Rev Cancer 17, 223-238 (2017).
4 Han et al. Am J Hum Genet 106, 202-214 (2020).
5 Serpas et al. PNAS 116, 641-649 (2019).
6 Heitzer et al. Trends Mol Med 26, 519-528 (2020).
7 Kustanovich et al. Cancer Biol Ther 20, 1057-1067 (2019).
8 Teif & Clarkson, in Encyclopedia of Bioinf and Comp Biology, 308-317 (Academic Press, Oxford, 2019).
9 Clarkson et al. Nucleic Acids Res 47, 11181-11196 (2019).
10 Teif, V. B. et al. Nat Struct Mol Biol 19, 1185-92 (2012).
11 Wiehle et al. Genome Res 29, 750-761 (2019).
12 Teif & Rippe. Nucleic Acids Res 37, 5641-55 (2009).
13 Teif et al. Nucleus 8, 188-204 (2017).
14 Mallm et al. Mol Syst Biol 15, e8339 (2019).
15 Kitzman et al. Sci Transl Med 4, 137ra76 (2012).
16 Sun et al. PNAS 115, E5106-e5114 (2018).
17 Phallen et al. Sci Transl Med 9 (2017).
18 Zviran et al. Nat Med 26, 1114-1124 (2020).
19 Cristiano et al. Nature 570, 385-389 (2019).
20 Frenel et al. Clin Cancer Res 21, 4586-96 (2015).
21 Dwivedi et al. Crit Care 16, R151 (2012).
22 Cheng et al. Med (N Y) (2021).
23 Abbosh et al. Nature 545, 446-451 (2017).
24 Wan et al. BMC Cancer 19, 832 (2019).
25 Dudley & Diehn, Annu Rev Pathol (2020).
26 Palande et al. bioRxiv, 2020.02.25.963975 (2020).
27 Mouliere et al. EMBO Mol Med 10 (2018).
28 van der Pol & Mouliere. Cancer Cell 36, 350-368 (2019).
29 Nassiri et al. Nature Medicine 26, 1044-1047 (2020).
30 Shen et al. Nature 563, 579-583 (2018).
31 Liu et al. Annals of Oncology 31, 745-759 (2020).
32 Erger et al. Genome Med 12, 54 (2020).
33 Song et al. Cell Research 27, 1231-1242 (2017).
34 Im et al. Trends Cancer (2020).
35 Underhill et al. PLoS Genet 12, e1006162 (2016).
36 Guo et al. BMC Genomics 21, 473 (2020).
37 Markus et al. bioRxiv, 696633 (2019).
38 Mouliere et al. Sci Transl Med 10 (2018).
39 Snyder et al. Cell 164, 57-68 (2016).
40 Zukowski et al. Open Bio/10, 200119 (2020).
41 Chandrananda et al. BMC Med Genomics 8, 29 (2015).
42 Wong et al. Nat Med 21, 815-9 (2015).
43 Rostami et al. Cell Rep 31, 107830 (2020).
44 Wan et al. BMC Cancer 19, 832 (2019).
45 Vainshtein et al. BMC Genomics 18, 158 (2017).

1. A method for identifying genomic regions with condition-sensitive occupancy of nucleosomes and/or chromatin macromolecules, the method comprising:
(a) comparing, to a reference genome sequence, at least a portion of:
(i) a plurality of first nucleic acid sequence datasets, each first nucleic acid sequence dataset being obtained from a plurality of digestion-protected regions of a plurality of nucleic acid molecules obtained from a first subject with a first condition, wherein the plurality of first nucleic acid sequence datasets each comprise a plurality of first nucleic acid fragments;
wherein a genomic location of each of the plurality of first nucleic acid fragments is identified;
(b) comparing, to the reference genome sequence, at least a portion of:
(i) a plurality of second nucleic acid sequence datasets, each second nucleic acid sequence dataset being obtained from a plurality of digestion-protected regions of a plurality of nucleic acid molecules obtained from a second subject with a second condition, wherein the plurality of second nucleic acid sequence datasets each comprise a plurality of second nucleic acid fragments;
wherein a genomic location of each of the plurality of second nucleic acid fragments is identified;
(c) (i) determining an average normalised occupancy of digestion-protected regions of nucleic acid fragments per genomic region (O₁) of each of the first subjects with the first condition and (ii) determining an average normalised occupancy of digestion-protected regions of nucleic acid fragments per genomic region (O₂) of each of the second subjects with the second condition; wherein the method optionally comprises repeating step (c) for one or more other conditions (N) to determine average normalised occupancy of digestion-protected regions of nucleic acid fragments per genomic region (O_{N}) of subjects with condition N;
(d) determining one or more stable-nucleosome regions of the genome in which the variation of the occupancy of digestion-protected regions of nucleic acid fragments in each of the first subjects with the first condition is below a first set threshold value;
(e) determining one or more stable-nucleosome regions of the genome in which the variation of the occupancy of digested-protected regions of nucleic acid fragments in each of the second subjects with the second condition is below a second set threshold value;
(f) comparing (i) the one or more stable-nucleosome regions of the genome of the first subjects with the first condition and (ii) the one or more stable-nucleosome regions of the genome of the second subjects with the second condition; and
(g) identifying one or more regions of the genome which have stable-nucleosome regions in the first condition and stable-nucleosome regions in the second condition, and which have a difference between the average normalised occupancy of digestion-protected regions of nucleic acid fragments in the first condition and the average normalised occupancy of digestion-protected regions of nucleic acid fragments in the second condition that is larger or smaller than a set threshold value, to thereby identify one or more condition-sensitive-genomic regions.

2. The method according to claim 1, wherein the stable nucleosome region is a stable-nucleosome-occupancy region.

3. The method according to claim 1, wherein step (d) comprises (ii) determining one or more fuzzy-nucleosome regions of the genome in which the variation of the occupancy of the nucleic acid fragments in digestion-protected regions in each of the first subjects with the first condition is above a first set threshold value.

4. (canceled)

5. (canceled)

6. A method for identifying genomic regions with condition-sensitive positioning of nucleosomes and/or chromatin macromolecules, the method comprising:
(a) comparing, to a reference genome sequence, at least a portion of:
(i) a plurality of first nucleic acid sequence datasets, each first nucleic acid sequence dataset being obtained from a plurality of digestion-protected regions of a plurality of nucleic acid molecules obtained from a first subject with a first condition, wherein the plurality of first nucleic acid sequence datasets each comprise a plurality of first nucleic acid fragments;
wherein a genomic location of each of the plurality of first nucleic acid fragments is identified;
(b) comparing, to the reference genome sequence, at least a portion of:
(i) a plurality of second nucleic acid sequence datasets, each second nucleic acid sequence dataset being obtained from a plurality of digestion-protected regions of a plurality of nucleic acid molecules obtained from a second subject with a second condition, wherein the plurality of second nucleic acid sequence datasets each comprise a plurality of second nucleic acid fragments;
wherein a genomic location of each of the plurality of second nucleic acid fragments is identified;
(c) (i) determining the genomic locations defined by the region start and region end coordinates of digestion-protected regions of nucleic acid fragments of each of the first subjects with the first condition and (ii) determining the genomic locations of digestion-protected regions of nucleic acid fragments of each of the second subjects with the second condition; wherein the method optionally comprises repeating step (c) for one or more other conditions (N) to determine the genomic locations of digestion-protected regions of nucleic acid fragments of subjects with condition N;
(d) determining one or more stable-nucleosome-positioning regions of the genome in which the variation of the genomic locations defined by the start and end coordinates or the center coordinates of digestion-protected regions of nucleic acid fragments in each of the first subjects with the first condition is below a first set threshold value;
(e) determining one or more stable-nucleosome-positioning regions of the genome in which the variation of the genomic locations defined by the start and end coordinates or the center coordinates of digested-protected regions of nucleic acid fragments in each of the second subjects with the second condition is below a second set threshold value;
(f) comparing (i) the one or more stable-nucleosome-positioning regions of the genome of the first subjects with the first condition and (ii) the one or more stable-nucleosome-positioning regions of the genome of the second subjects with the second condition;
(g) identifying one or more condition-sensitive regions of the genome which have stable-nucleosome-positioning regions in the first condition and which changed their genomic locations in the second condition by a value larger or smaller than a set threshold value, to thereby identify one or more condition-sensitive genomic regions where the nucleosome locations shifted to form the dataset of shifted nucleosomes;
(h) identifying one or more condition-sensitive regions of the genome which have stable-nucleosome-positioning regions in the first condition and which do not overlap with stable-nucleosome-positioning regions in the second condition, to thereby identify one or more condition-sensitive genomic regions that preferentially contain a nucleosome in the first condition and preferentially lose nucleosomes in the second condition to form the dataset of lost nucleosomes; and
(i) identifying one or more condition-sensitive regions of the genome which have stable-nucleosome-positioning regions in the second condition and which do not overlap with stable-nucleosome-positioning regions in the first condition, to thereby identify one or more condition-sensitive genomic regions that preferentially do not contain a nucleosome in the first condition and which gained nucleosomes in the second condition to form the dataset of gained nucleosomes.

7. The method according to claim 1, which further comprises:
identifying one or more regions of the genome which comprise condition-sensitive regions for combinations of different conditions by determining intersections, unions and/or exclusions of condition-sensitive regions, wherein:
the condition-sensitive regions comprise regions with changed DNA protection by nucleosomes and/or other chromatin complexes according to claim 1 or regions with changed nucleosome positioning according to claim 6;
intersections define regions sensitive to each of a plurality of conditions,
unions are composed of condition-sensitive regions defined for more than two pairs of conditions of interest; wherein unions define regions sensitive to at least one of a plurality of conditions; and
exclusions define regions sensitive to a set of conditions but not sensitive to a differing set of conditions; and
refining the set of condition-sensitive-nucleosome genomic regions by including or excluding condition-sensitive-genomic regions defined for comorbidities such as ageing.

8.-11. (canceled)

12. The method according to claim 1, wherein step (c) comprises splitting the reference genomic sequence into regions of a predetermined length and determining average normalised occupancy of protected regions of nucleic acid fragments within each region.

13. The method according to claim 12, wherein the sizes of the genomic regions for the calculation of normalised occupancy are between 10 base pairs (bp) and 100000 bp in length, optionally wherein the regions are 50-150 bp in length, wherein optionally the sizes of the genomic regions for the calculation of normalised occupancy are between 10 base pairs (bp) and 10000 bp in length.

14. (canceled)

15. (canceled)

16. The method according to claim 1, which comprises applying a pairwise dissimilarity threshold value defining a minimal acceptable value of the relative difference of an average normalised occupancy of protected regions of nucleic acid fragments in the first condition (O₁) and the second condition (O₂), wherein the relative difference is defined as (O₂−O₁)/(01+Oz).

17. The method according to claim 1, wherein the condition-sensitive region of the genome comprises a difference between the subjects with the first condition and the subjects with the second condition in one or more of the following, or in a combination thereof:
(i) average profile of the occupancy of protected regions of nucleic acid fragments;
(ii) genomic location of the center of nucleosome;
(iii) genomic locations of the start and end of the nucleosome;
(iv) size of linker DNA between nucleosomes;
(v) stability of nucleosomes against digestion by MNase or another nuclease;
(vi) stability of the nucleosome against partial DNA unwrapping;
(vii) stability of the nucleosome against partial disassembly of the histone octamer;
(viii) accessibility of DNA as measured by ATAC-seq or/and DNase-seq; and/or
(ix) protein binding as measured by ChIP-seq or CUT&RUN or CUT&Tag.

18. The method according to claim 1, which further comprises, prior to step (a) and/or step (b):
(i) obtaining first nucleic acid sequence data from the digestion-protected regions of the nucleic acid molecules from a plurality of subjects with the first condition, wherein the first nucleic acid sequence data comprises a plurality of first nucleic acid fragments; and/or
(ii) obtaining second nucleic acid sequence data obtained from digestion-protected regions of nucleic acid molecules from a plurality of subjects with the second condition, wherein the second nucleic acid sequence data comprises a plurality of second nucleic acid fragments.

19.-29. (canceled)

30. The method according to claim 1, wherein the first condition is a pathological disorder selected from a cancer, a sub-type of cancer, a viral infection, a bacterial infection, an inflammatory disorder, sepsis, cardiovascular disorder, acute cellular rejection, benign kidney disease, benign liver disease, hepatitis B, inflammatory bowel disease, lupus, diabetes, Crohn's disease, myocarditis, pericarditis, multiple sclerosis, psoriasis and a neurological disease.

31. (canceled)

32. (canceled)

33. The method according to claim 1, wherein the second condition is the absence of a pathological disorder.

34. (canceled)

35. The method according to claim 1, wherein the first condition is a pathological disorder and the second condition is the absence of the pathological disorder.

36.-40. (canceled)

41. A system for identifying condition-sensitive regions in cell-free DNA, the system comprising a computer program configured to;
(a) compare, to a reference genome sequence, at least a portion of:
(i) a plurality of first nucleic acid sequence datasets, each first nucleic acid sequence dataset being obtained from a plurality of digestion-protected regions of a plurality of nucleic acid molecules obtained from a first subject with a first condition, wherein the plurality of first nucleic acid sequence datasets each comprise a plurality of first nucleic acid fragments;
wherein a genomic location of each of the plurality of first nucleic acid fragments is identified;
(b) compare, to the reference genome sequence, at least a portion of:
(i) a plurality of second nucleic acid sequence datasets, each second nucleic acid sequence dataset being obtained from a plurality of digestion-protected regions of a plurality of nucleic acid molecules obtained from a second subject with a second condition, wherein the plurality of second nucleic acid sequence datasets each comprise a plurality of second nucleic acid fragments;
wherein a genomic location of each of the plurality of second nucleic acid fragments is identified;
(c) (i) determine an average normalised occupancy of digestion-protected regions of nucleic acid fragments per genomic region (O₁) of the first subjects with the first condition and (ii) determine an average occupancy of digestion-protected regions of nucleic acid fragments per genomic region (O₂) of the second subjects with the second condition; optionally repeat step (c) for any other condition N to determine average normalised occupancy of protected regions of nucleic acid fragments per genomic region (O_{N}) of subjects with condition N;
(d) determine one or more stable-nucleosome regions of the genome in which the variation of the occupancy of protected regions of nucleic acid fragments in each of the first subjects with the first condition is below a first set threshold value;
(e) determine one or more stable-nucleosome regions of the genome in which the variation of the occupancy of protected regions of nucleic acid fragments in each of the second subjects with the second condition is below a second set threshold value;
(f) compare (i) the one or more stable-nucleosome regions of the genome of the first subjects with the first condition and (ii) the one or more stable-nucleosome regions of the genome of the second subjects with the second condition; and
(g) identify one or more regions of the genome which have stable-nucleosome regions in the first condition and stable-nucleosome regions in the second condition, and which have the difference between the average occupancy of protected regions of nucleic acid fragments in the first condition and the average occupancy of protected regions of nucleic acid fragments in the second condition larger or smaller than set threshold values, to thereby identify one or more condition-sensitive regions.

42. The system according to claim 41, wherein the stable nucleosome region is a stable-nucleosome-occupancy region.

43. The system according to claim 41, wherein step (d) comprises (ii) determining one or more fuzzy-nucleosome regions of the genome in which the variation of the occupancy of the nucleic acid fragments in digestion-protected regions in each of the first subjects with the first condition is above a first set threshold value.

44. (canceled)

45. (canceled)

46. A system for identifying condition-sensitive regions in cell-free DNA, the system comprising a computer program configured to:
(a) comparing, to a reference genome sequence, at least a portion of:
(i) a plurality of first nucleic acid sequence datasets, each first nucleic acid sequence dataset being obtained from a plurality of digestion-protected regions of a plurality of nucleic acid molecules obtained from a first subject with a first condition, wherein the plurality of first nucleic acid sequence datasets each comprise a plurality of first nucleic acid fragments;
wherein a genomic location of each of the plurality of first nucleic acid fragments is identified;
(b) comparing, to the reference genome sequence, at least a portion of:
(i) a plurality of second nucleic acid sequence datasets, each second nucleic acid sequence dataset being obtained from a plurality of digestion-protected regions of a plurality of nucleic acid molecules obtained from a second subject with a second condition, wherein the plurality of second nucleic acid sequence datasets each comprise a plurality of second nucleic acid fragments;
wherein a genomic location of each of the plurality of second nucleic acid fragments is identified;
(c) (i) determining the genomic locations defined by the region start and region end coordinates of digestion-protected regions of nucleic acid fragments of each of the first subjects with the first condition and (ii) determining the genomic locations of digestion-protected regions of nucleic acid fragments of each of the second subjects with the second condition; wherein the method optionally comprises repeating step (c) for one or more other conditions (N) to determine the genomic locations of digestion-protected regions of nucleic acid fragments of subjects with condition N;
(d) determining one or more stable-nucleosome-positioning regions of the genome in which the variation of the genomic locations defined by the start and end coordinates of digestion-protected regions of nucleic acid fragments in each of the first subjects with the first condition is below a first set threshold value;
(e) determining one or more stable-nucleosome-positioning regions of the genome in which the variation of the genomic locations defined by the start and end coordinates of digested-protected regions of nucleic acid fragments in each of the second subjects with the second condition is below a second set threshold value;
(f) comparing (i) the one or more stable-nucleosome-positioning regions of the genome of the first subjects with the first condition and (ii) the one or more stable-nucleosome-positioning regions of the genome of the second subjects with the second condition;
(g) identifying one or more condition-sensitive regions of the genome which have stable-nucleosome-positioning regions in the first condition and which changed their genomic locations in the second condition by a value larger or smaller than a set threshold value, to thereby identify one or more condition-sensitive genomic regions where the nucleosome locations shifted (“shifted nucleosomes”);
(h) identifying one or more condition-sensitive regions of the genome which have stable-nucleosome-positioning regions in the first condition and which do not overlap with stable-nucleosome-positioning regions in the second condition, to thereby identify one or more condition-sensitive genomic regions that preferentially contain a nucleosome in the first condition and preferentially lost nucleosome in the second condition (“lost nucleosomes”); and
(i) identifying one or more condition-sensitive regions of the genome which have stable-nucleosome-positioning regions in the second condition and which do not overlap with stable-nucleosome-positioning regions in the first condition, to thereby identify one or more condition-sensitive genomic regions that preferentially do not contain a nucleosome in the first condition and gained nucleosome in the second condition (“gained nucleosomes”).

47. The system according to claim 41, which is further configured to:
(h) identify one or more regions of the genome which comprise condition-sensitive regions for combinations of different conditions by determining intersections, unions or exclusions of condition-sensitive regions, where intersections define regions sensitive to each of several conditions of interest, unions define regions sensitive to at least one of several conditions of interest and exclusions define regions sensitive to some conditions but not sensitive to other specified conditions (for example, sensitive to cancer but not sensitive to ageing); and
refine the set of condition-sensitive genomic regions by including or excluding condition-sensitive regions defined for comorbidities.

48. A method of identifying a condition in a subject, the method comprising:
(a) defining one or more characteristics for a set of condition-specific regions;
(b) defining the set of condition-specific regions by performing a method for identifying genomic regions with condition-sensitive occupancy or positioning of nucleosomes and/or chromatin macromolecules as claimed in claim 1;
(c) obtaining nucleic acid sequence data from at least a portion of cell free DNA (cfDNA) isolated from a sample derived from the subject, wherein the subject is a first subject in which a condition is to be determined;
(d) performing an alignment of sequenced data to a reference genome to define the genomic coordinates of sequenced reads;
(e) calculating a normalised occupancy of cfDNA per genomic region, separately for each sample;
(f) creating a reference set of samples, each of which are known to be obtained from a subject having a predetermined condition;
(g) calculating an average normalised occupancy of cfDNA, separately for each sample in the reference set of step (f) for each condition-specific region;
(h) performing dimensionality reduction analysis on (1) the sample obtained from the first subject in which the condition needs to be determined and (2) the samples from the reference set of samples; and
(i) performing a classification of the sample from the first subject based on the similarity of the average normalised cfDNA occupancy in condition-sensitive regions to clusters formed by the samples from the reference set.

49. (canceled)

50. The method according to claim 48, wherein the normalisation is performed by dividing the number of protected regions of nucleic acid fragments in a predetermined genomic region by the average occupancy for a predetermined genomic region in a predetermined sample in a predetermined condition.

51.-54. (canceled)
